(19) United States
(12) Patent Application Publication
(10) Pub. No.: US 2026/0176376 A1
(43) Pub. Date: Jun. 25, 2026

(54) METHODS FOR PRODUCING CELLS

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Michel Sadelain, New York, NY (US); Isabelle RIVIERE, New York, NY (US); Xiuyan WANG, New York, NY (US); Sascha P. HAUBNER, New York, NY (US); Anthony DANIYAN, New York, NY (US)

(21) Appl. No.: 19/532,616

(22) Filed: Feb. 6, 2026

Related U.S. Application Data
(63) Continuation of application No. PCT/US2023/029609, filed on Aug. 7, 2023.

(51) Int. Cl. C07K 16/32; A61K 40/31

(52) U.S. Cl. CPC C07K 16/32; A61K 40/31

(57) ABSTRACT

The presently disclosed subject matter provides methods for improving production of cells comprising an antigen-recognizing receptor (e.g., a chimeric antigen receptor (CAR) or a TCR like fusion molecule). The methods disclosed herein can improve the activity and/or efficiency of the cells.

### CROSS-REFERENCE TO RELATED APPLICATION

This application is a Continuation Application of International Patent Application No. PCT/US2023/029609, filed on Aug. 7, 2023, the content of which is hereby incorporated by reference in its entirety, and to which priority is claimed.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Feb. 6, 2026, is entitled 0893390363_ST26.xml, and is 90,076 bytes in size.

### 1. INTRODUCTION

The presently disclosed subject matter provides methods and compositions for enhancing the production of cells comprising an antigen-recognizing receptor (e.g., a chimeric antigen receptor (CAR) or a TCR like fusion molecule). The methods and compositions disclosed herein can improve the activity and/or efficiency of the cells.

### 2. BACKGROUND OF THE INVENTION

With the US Food and Drug Administration (FDA) approval of four CD19-and one BCMA-targeted chimeric antigen receptor (CAR) therapy for B cell malignancies, CAR T cell therapy has finally reached the status of a medicinal product. The successful manufacturing of autologous CAR T cell products is a key requirement for this promising treatment modality. Recently, it was found that high CD14⁺ cell content poses a challenge for manufacturing CAR T cells, especially in patients with non-Hodgkin's lymphoma and multiple myeloma caused by the non-specific phagocytosis of the magnetic beads used to activate CD3⁺ T cells (Wang et al., Mol Ther Methods Clin Dev. 2021 Jul. 16; 22:377-387). Therefore, there remains a need in the art for improved manufacturing processes.

### 3. SUMMARY OF THE INVENTION

The presently disclosed subject matter provides improved methods and composition useful to generate cells with enhanced activity and efficacy for immunotherapy (e.g., T cell immunotherapy). In certain non-limiting embodiments, the presently disclosed subject matter provides a method of producing an immunoresponsive cell comprising an antigen-recognizing receptor, the method comprising a) positively selecting CD3⁺ immunoresponsive cells solely with a CD3⁺ monospecific reagent; b) obtaining a composition enriched for CD3⁺ immunoresponsive cells; c) contacting the CD3⁺ immunoresponsive cells with a reagent activating and/or stimulating the immunoresponsive cells; and d) introducing an antigen-recognizing receptor into the immunoresponsive cells.

In certain embodiments, the CD3⁺ monospecific reagent is an anti-CD3 antibody or an antigen-binding fragment thereof. In certain embodiments, the anti-CD3 antibody or an antigen-binding fragment thereof does not activate the immunoresponsive cell. In certain embodiments, the anti-CD3 antibody or an antigen-binding fragment thereof binds to an epitope located on the human CD3ε chain.

In certain embodiments, the anti-CD3 antibody or antigen-fragment thereof is bound to a magnetic particle. In certain embodiments, the anti-CD3 antibody or antigen-fragment thereof is bound to an iron-dextran particle. In certain embodiments, the magnetic particle has a diameter of from about 10 nm to about 100 nm. In certain embodiments, the magnetic particle has a diameter of about 50 nm. In certain embodiments, the CD3⁺ monospecific reagent is contacted with a sample at a cell density of about 100×10⁶ cells/ml.

In certain embodiments, the positive selection is a chromatography-based selection. In certain embodiments, the composition comprises at least about 80%, at least about 85%, at least about 90%, at least about 95%, at least about 96%, at least about 97%, at least about 98%, at least about 99%, or about 100% more CD3⁺ immunoresponsive cells compared to a sample. In certain embodiments, the composition comprises at least about 90% more CD3⁺ immunoresponsive cells compared to the sample. In certain embodiments, the composition comprises at least about 60%, at least about 65%, at least about 70%, at least about 75%, at least about 80%, at least about 85%, at least about 90%, at least about 95%, at least about 96%, at least about 97%, at least about 98%, at least about 99%, or about 100% of CD3⁺ immunoresponsive cells over the total number of cells. In certain embodiments, the composition comprises at least about 90% of CD3⁺ immunoresponsive cells over the total number of cells.

In certain embodiments, the composition comprises at least about 80%, at least about 85%, at least about 90%, at least about 95%, at least about 96%, at least about 97%, at least about 98%, at least about 99%, or about 100% less CD14⁺ immunoresponsive cells compared to the sample. In certain embodiments, the composition comprises at least about 80% less CD14⁺ immunoresponsive cells compared to a sample. In certain embodiments, the composition comprises at least about 1%, at least about 5%, at least about 10%, at least about 15%, or at least about 20% of CD14⁺ immunoresponsive cells over the total number of cells. In certain embodiments, the composition comprises at least about 5% of CD14⁺ immunoresponsive cells over the total number of cells.

In certain embodiments, the reagent activating and/or stimulating the immunoresponsive cells comprises an anti-CD3 antibody or an antigen-binding fragment thereof activating the immunoresponsive cell. In certain embodiments, the reagent activating and/or stimulating the immunoresponsive cells comprises an anti-CD28 antibody or an antigen-binding thereof. In certain embodiments, the reagent activating and/or stimulating the immunoresponsive cells comprises an anti-CD3 antibody or an antigen-binding fragment thereof activating the immunoresponsive cell and an anti-CD28 antibody or an antigen-binding thereof. In certain embodiments, the reagent is bound to a magnetic particle.

In certain embodiments, the immunoresponsive cell are T cells. In certain embodiments, the T cells are selected from the group consisting of cytotoxic T lymphocytes (CTL), γδ T cells, tumor-infiltrating lymphocytes (TIL), regulatory T cells, and Natural Killer T (NKT) cells. In certain embodiments, the T cells are CD8⁺ T cells. In certain embodiments, the T cells are CD4⁺ T cells. In certain embodiments, the CD8⁺ T cells are CD4 independent. In certain embodiments, the immunoresponsive cells are CD14⁻ cells.

In certain embodiments, the immunoresponsive cells are obtained from a sample selected from the group consisting of a blood sample, a blood-derived sample, an apheresis product, a leukapheresis product, a whole blood sample, a peripheral blood mononuclear cells (PBMCs) sample, a leukocytes sample, a bone marrow sample, a thymus sample, a tissue biopsy sample, a tumor sample, a leukemia sample, a lymphoma sample, a lymph node sample, a gut associated lymphoid tissue sample, a mucosa associated lymphoid tissue sample, a spleen sample, a liver sample, a lung sample, a stomach sample, an intestine sample, a colon sample, a kidney sample, a pancreas sample, a breast sample, a bone sample, a prostate sample, a cervix sample, a testes sample, an ovaries sample, a tonsil sample, and a combination thereof. In certain embodiments, the sample is an apheresis product. In certain embodiments, the sample is a leukapheresis product. In certain embodiments, the immunoresponsive cells are obtained from a sample of a subject having acute myeloid leukemia (AML).

In certain embodiments, the method further comprises a density-based cell separation. In certain embodiments, the density-based cell separation allows purification of peripheral blood mononuclear cells (PBMCs).

In certain embodiments, the antigen-recognizing receptor targets an antigen. In certain embodiments, the antigen is a tumor antigen or a pathogen antigen. In certain embodiments, the tumor antigen is selected from the group consisting of CD19, CD70, IL1RAP, ABCG2, AChR, ACKR6, ADAMTS13, ADGRE2 (EMR2), ADORA3, ADRAID, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLECIA, CLL1, CNIH2, COL15A1, COLEC12, CPM, CR1, CX3CR1, CXCR4, CYP4F11, DAGLB, DARC, DFNB31, DGKI, EGFIR, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGRIA, FKBPIB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, K-light chain, LICAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNNID, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSFIB, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the antigen-recognizing receptor is a chimeric receptor, a T cell receptor (TCR), or a TCR like fusion molecule. In certain embodiments, the antigen-recognizing receptor is a chimeric receptor. In certain embodiments, the chimeric receptor is a chimeric antigen receptor (CAR).

In certain embodiments, the CAR comprises an extracellular antigen-binding domain that binds to the antigen, and an intracellular signaling domain that is capable of delivering an activation signal to the cell. In certain embodiments, the intracellular signaling domain of the CAR comprises a CD3ζ polypeptide. In certain embodiments, the CD3ζ polypeptide is a native CD3ζ polypeptide or a modified CD3ζ polypeptide. In certain embodiments, the modified CD3ζ polypeptide comprises a native ITAM1, an ITAM2 variant consisting of two loss-of-function mutations, and an ITAM3 variant consisting of two loss-of-function mutations. In certain embodiments, the intracellular signaling domain of the CAR further comprises at least one costimulatory signaling region. In certain embodiments, the at least one costimulatory signaling region comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D. In certain embodiments, the CAR comprises a transmembrane domain.

In certain embodiments, the chimeric receptor is a chimeric co-stimulating receptor (CCR). In certain embodiments, wherein the CCR comprises an extracellular antigen-binding domain that binds to the second antigen and an intracellular domain that is capable of delivering a co-stimulatory signal to the cell but does not alone deliver an activation signal to the cell. In certain embodiments, the intracellular domain of the CCR comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the antigen-recognizing receptor is a TCR like fusion molecule. In certain embodiments, the TCR like fusion molecule comprises i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise a TRAC polypeptide or a TRBC polypeptide, and b) bind to the first antigen, wherein the TCR like fusion molecule binds to the first antigen in an HLA-independent manner. In certain embodiments, at least one of the TRAC polypeptide and the TRBC polypeptide is endogenous. In certain embodiments, the antigen-binding chain comprises an antigen-binding fragment of a VH of an antibody and a TRBC polypeptide, and the second antigen-binding chain comprises an antigen-binding fragment of a VL of the antibody and a TRAC polypeptide. In certain embodiments, the antigen-binding chain comprises an antigen-binding fragment of a VH of an antibody and a TRAC polypeptide, and the second antigen-binding chain comprises an antigen-binding fragment of a VL of the antibody and a TRBC polypeptide. In certain embodiments, the first and second antigen binding chains are capable of associating with a CD3ζ polypeptide. In certain embodiments, the first and second antigen binding chains, upon binding to the first antigen, are capable of activating the CD3% polypeptide.

In certain non-limiting embodiments, the presently disclosed subject matter also provides a composition comprising the immunoresponsive cell obtained by the methods disclosed herein. In certain embodiments, the composition is a pharmaceutical composition further comprising a pharmaceutically acceptable excipient.

In certain non-limiting embodiments, the presently disclosed subject matter further provides a method of reducing tumor burden in a subject, preventing and/or treating a neoplasm or a tumor in the subject, preventing and/or treating a pathogen infection in a subject, preventing and/or treating an autoimmune disease in a subject, and/or preventing and/or treating an infectious disease in a subject. In certain embodiments, the method comprises administering to the subject an effective amount of the immunoresponsive cell produced by the method or the composition disclosed herein.

In certain embodiments, the method reduces the number of tumor cells, reduces tumor size, and/or eradicates the tumor in the subject. In certain embodiments, the neoplasm or tumor is cancer. In certain embodiments, the neoplasm or tumor is a solid tumor. In certain embodiments, the neoplasm or tumor is a blood cancer. In certain embodiments, the neoplasm or tumor is a myeloid disorder. In certain embodiments, the myeloid disorder is selected from the group consisting of myelodysplastic syndromes, myeloproliferative neoplasms, chronic myelomonocytic leukemia, or acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera. In certain embodiments, the myeloid disorder is acute myeloid leukemia (AML).

Further, in certain non-limiting embodiments, the presently disclosed subject matter provides a kit comprising the immunoresponsive cell produced by the method or the composition disclosed herein.

In certain embodiments, the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.

In certain embodiments, the composition or the kit disclosed herein are for use in reducing tumor burden, treating and/or preventing a neoplasm or a tumor, preventing and/or treating a pathogen infection, preventing and/or treating an autoimmune disease, and/or preventing and/or treating an infectious disease, in a subject.

### 4. BRIEF DESCRIPTION OF THE FIGURES

The following Detailed Description, given by way of example, but not intended to limit the presently disclosed subject matter to specific embodiments described, may be understood in conjunction with the accompanying drawings.

FIG. 1 illustrates an exemplary schematic of the selection procedure disclosed herein.

FIGS. 2A-2C illustrate the cytotoxicity of CAR-T cells generated from CD3⁺ selected cells or CD4⁺/CD8⁺ selected cells. FIG. 2A shows FACS analysis of chimeric receptors expressed CD3⁺ selected cells or CD4⁺/CD8⁺ selected cells. FIG. 2B shows EM/CM phenotype of CAR-T cells generated from CD3⁺ selected cells or CD4⁺/CD8⁺ selected cells. FIG. 2C shows the killing activity of CD3⁺ selected cells or CD4⁺/CD8⁺ selected cells in in vitro cytotoxic assay.

FIGS. 3A and 3B illustrate in vivo antitumor activity of CAR-T cells generated from CD3⁺ selected cells or CD4⁺/CD8⁺ selected cells. FIG. 3A shows a schematic of the in vivo experiments. FIG. 3B shows the total flux and survival curves of CD3⁺ selected cells or CD4⁺/CD8⁺ selected cells expressing an ADCLEC.syn CAR/CCR system.

### 5. DETAILED DESCRIPTION OF THE INVENTION

The presently disclosed subject matter provides improved methods and composition useful to generate cells with enhanced activity and efficacy for immunotherapy (e.g., T cell immunotherapy). The presently disclosed subject matter is based, in part, on the unexpected discovery that positively selected CD3⁺ T cells can be used to manufacture potent CAR T cells. Surprisingly, the presently disclosed methods do not induce tolerance and/or anergy of the T cells. The presently disclosed methods also allow the removal of certain tumor cells (e.g., AML tumor cells, CD14+ cells). In certain embodiments, the presently disclosed methods can be used to manufacture CAR-T cells for AML.

Non-limiting embodiments of the presently disclosed subject matter are described by the present specification and Examples.

For purposes of clarity of disclosure and not by way of limitation, the detailed description is divided into the following subsections:
5.1 Definitions;
5.2. Manufacturing Methods;
5.3. Cells;
5.4. Formulations and Administration;
5.5. Methods of Treatment;
5.6. Kits; and
5.7. Exemplary Embodiments.

### 5.1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, a “co-stimulatory molecule” refers to a cell surface molecule other than an antigen receptor or its ligand that can provide an efficient response of lymphocytes to an antigen. In certain embodiments, a co-stimulatory molecule can provide optimal lymphocyte activation.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage. I n certain embodiments, the immunoresponsive cell is a monocyte.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane-bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40, ICOS, DAP-10, CD27, NKG2D, CD2, CD150, CD226. Receiving multiple stimulatory signals can be important to mount a robust and long-term T cell mediated immune response. T cells can quickly become inhibited and unresponsive to antigens. While the effects of these co-stimulatory signals may vary, they generally result in increased gene expression in order to generate long lived, proliferative, and anti-apoptotic T cells that robustly respond to antigens for complete and sustained eradication.

The term “antigen-recognizing receptor” as used herein refers to a receptor that is capable of activating an immune or immunoresponsive cell (e.g., a T-cell) in response to its binding to an antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain variable fragment (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H}1, C_{H}2 and C_{H}3. Each light chain is comprised of a light chain variable region (abbreviated herein as V_{L}) and a light chain constant C_{L} region. The light chain constant region is comprised of one domain, C_{L}. The VHand V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs regions are delineated using the PyIgClassify system (Adolf-Bryfogle et al., Nucleic acids research 43.D1 (2015): D432-D438).

As used herein, the term “Linker” shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker is a G4S linker. In certain embodiments, the linker comprises or consists of the amino acid sequence set forth in SEQ ID NO: 1, which is provided below:

[SEQ ID NO: 1]
GGGGSGGGGSGGGGS

In certain embodiments, the linker comprises or consists of the amino acid sequence set forth in SEQ ID NO: 2, which is provided below:

[SEQ ID NO: 2]
GGGGSGGGGSGGGSGGGGS

In certain embodiments, the linker comprises or consists of the amino acid sequence set forth in SEQ ID NO: 3, which is provided below:

[SEQ ID NO: 3]
GGGGSGGGGSGGGGSGGGSGGGGS

In certain embodiments, the linker comprises or consists of the amino acid sequence set forth in SEQ ID NO: 4, which is provided below:

[SEQ ID NO: 4]
GGGGSGGGGSGGGGSGGGGSGGGSGGGGS

In certain embodiments, the linker comprises or consists of the amino acid sequence set forth in SEQ ID NO: 5, which is provided below:

[SEQ ID NO: 5]
GGGGS

In certain embodiments, the linker comprises or consists of the amino acid sequence set forth in SEQ ID NO: 6, which is provided below:

[SEQ ID NO: 6]
GGGGSGGGGS

As used herein, the term “single-chain variable fragment” or “scFv” is a fusion protein of the variable regions of the heavy (V_{H}) and light chains (V_{L}) of an immunoglobulin covalently linked to form a V_{H}:: V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27 (6): 455-51; Peter et al., J Cachexia Sarcopenia Muscle 2012 Aug. 12; Shieh et al., J Imunol2009 183 (4): 2277-85; Giomarelli et al., Thromb Haemost 2007 97 (6): 955-63; Fife eta., J Clin Invst 2006 116 (8): 2252-61; Brocks et al., Immunotechnology 1997 3 (3): 173-84; Moosmayer et al., Ther Immunol 1995 2 (10:31-40). Agonistic scFvs having stimulatory activity have been described (see, e.g., Peter et al., J Bioi Chern 2003 25278 (38): 36740-7; Xie et al., Nat Biotech 1997 15 (8): 768-71; Ledbetter et al., Crit Rev Immunol1997 17 (5-6): 427-55; Ho et al., BioChim Biophys Acta 2003 1638 (3): 257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv703) disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed CD19-targeted CAR (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” is meant a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

By “specifically binds” is meant a polypeptide or a fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor or capable of suppressing an immune response via receptor-ligand binding.

As used herein, the term “monospecific reagent” refers to a substance (e.g., a small molecule, an antibody, etc.) that is only able to react with another single specified substance (e.g., an antigen or antigenic determinant). For example, but without any limitation, a monospecific reagent can be an antibody targeting a single antigen. For clarity, the term monospecific reagent does not encompass multispecific antibodies or reagents able to react with multiple substances (e.g., a combination of antibodies).

As used herein, the term “CD3⁺ monospecific reagent” refers to a substance that is only able to react with a CD3 polypeptide or a fragment thereof. For example, but without any limitations, a CD3⁺ monospecific reagent can be an antibody, an aptamer, a peptide, or a small molecule that binds to a CD3 polypeptide or a fragment thereof. For clarity, the term CD3⁺ monospecific reagent does not encompass antigen-presenting cells or reagents able to bind to a CD3 polypeptide and a CD28 polypeptide (e.g., a combination of antibodies targeting CD3 and CD28). Non-limiting examples of CD3⁺ monospecific reagent include Miltenyi™ CD3 MicroBeads and CliniMACS® CD3 GMP MicroBeads.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sports animals, rodents, and pets. Non-limiting examples of non-human animal subjects include rodents such as mice, rats, hamsters, and guinea pigs; rabbits; dogs, cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

Other aspects of the presently disclosed subject matter are described in the following disclosure and are within the ambit of the presently disclosed subject matter.

### 5.2. Manufacturing Methods

The presently disclosed subject matter provides methods and compositions for producing modified immunoresponsive cells. The presently disclosed methods are based, in part, on the discovery that positive selection of CD3⁺ immunoresponsive cells increases the purity of the cells used for manufacturing adoptive cell therapies (e.g., cells expressing a chimeric antigen receptor (CAR) or a TCR like fusion molecule, a HIT CAR), independently of their sources (e.g., healthy donors or cancer donors). Further, the positive selection of CD3⁺ immunoresponsive cells significantly reduced the amount of CD14⁺ cells (e.g., monocytes), which can negatively impact adoptive cell therapies in certain indications, e.g., acute myeloid leukemia (AML). In certain embodiments, the methods comprise contacting a sample with a CD3⁺ monospecific reagent to facilitate the positive selection of CD3⁺ immunoresponsive cells. In certain embodiments, the methods comprise contacting a sample with an anti-CD3 antibody or an antigen binding portion thereof to facilitate the positive selection of CD3⁺ immunoresponsive cells.

### 5.2.1. Samples

In certain embodiments, the presently disclosed subject matter provides methods of comprising contacting a sample with a reagent (e.g., a CD3⁺ monospecific reagent, e.g., an anti-CD3 antibody or antigen binding thereof) to select CD3⁺ immunoresponsive cells. In certain embodiments, the sample is a biological sample. In certain embodiments, the sample is selected from the group consisting of a blood sample, a blood-derived sample, an apheresis product, a leukapheresis product, a whole blood sample, a peripheral blood mononuclear cells (PBMCs) sample, a leukocytes sample, a bone marrow sample, a thymus sample, a tissue biopsy sample, a tumor sample, a leukemia sample, a lymphoma sample, a lymph node sample, a gut-associated lymphoid tissue sample, a mucosa-associated lymphoid tissue sample, a spleen sample, a liver sample, a lung sample, a stomach sample, an intestine sample, a colon sample, a kidney sample, a pancreas sample, a breast sample, a bone sample, a prostate sample, a cervix sample, a testes sample, an ovaries sample, a tonsil sample, and a combination thereof. In certain embodiments, the sample is an apheresis product. In certain embodiments, the sample is a leukapheresis product.

In certain embodiments, the sample is obtained by a subject having a tumor. For example, but without any limitation, the sample can be obtained by a subject having blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer.

In certain non-limiting embodiments, the sample is obtained by a subject having a tumor selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain non-limiting embodiments, the sample is obtained by a subject having a blood cancer selected from the group consisting of multiple myeloma, leukemia, lymphomas, acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, B cell prolymphocytic leukemia, Hodgkin's lymphoma, non-Hodgkin's lymphoma, B-cell non-Hodgkin's lymphoma, T-cell non-Hodgkin's lymphoma, B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, chronic lymphocytic leukemia/small lymphocytic lymphoma (CLL/SLL), B-cell prolymphocytic leukemia, splenic marginal zone lymphoma, hairy cell leukemia, splenic B-cell lymphoma/leukemia (unclassifiable), splenic diffuse red pulp small B-cell lymphoma, lymphoplasmacytic lymphoma, Waldenström macroglobulinemia, monoclonal gammopathy of undetermined significance (MGUS, IgM), heavy-chain diseases (μ, γ, α), MGUS (IgG/A), plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with IRF4 rearrangement, primary cutaneous follicle center cell lymphoma, mantle cell lymphoma, in situ mantle cell neoplasia, diffuse large B-cell lymphoma (DLBCL) (not otherwise specified (NOS)), germinal center B-cell type, activated B-cell type, T-cell/histiocyte-rich large B-cell lymphoma, primary DLBCL of the central nervous system (CNS), primary cutaneous DLBCL (leg type), Epstein-Barr virus (EBV)-positive DLBCL (NOS), EBV-positive mucocutaneous ulcer, DLBCL associated with chronic inflammation, lymphomatoid granulomatosis, primary mediastinal (thymic) large B-cell lymphoma, intravascular large B-cell lymphoma, anaplastic lymphoma kinase (ALK)-positive large B-cell lymphoma, plasmablastic lymphoma, primary effusion lymphoma, human herpesvirus 8 (HHV-8)-associated DLBCL (NOS), Burkitt lymphoma, Burkitt-like lymphoma with 11q aberration, high-grade B-cell lymphoma with MYC and BLC2 and/or BCL6 rearrangements, high-grade B-cell lymphoma (NOS), B-cell lymphoma (unclassifiable), myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

In certain non-limiting embodiments, the sample is obtained by a subject having acute myeloid leukemia (AML).

Apheresis and leukapheresis products can include several types of circulating cells (e.g., T cells, monocytes, granulocytes, B cells, other nucleated white blood cells, erythrocytes, and platelets). In certain embodiments, the presently disclosed methods include a preparation step. For example, but without any limitation, the preparation step can include washing, centrifugation, incubation, or a combination thereof. In certain non-limiting embodiments, the preparation step can include lysis of certain unwanted cells (e.g., erythrocytes) or separation of cells based on their density.

In certain embodiments, the preparation step comprises a density-based cell separation. In certain embodiments, the density-based cell separation includes centrifugation through a Percoll gradient. In certain embodiments, the density-based cell separation includes centrifugation through a Ficoll gradient. In certain embodiments, the density-based cell separation allows the purification of peripheral blood mononuclear cells (PBMCs).

### 5.2.2. Selection of CD3⁺ Immunoresponsive Cells

In certain non-limiting embodiments, the presently disclosed methods include a selection of cells based on the expression of certain markers (e.g., CD3, CD4, CD8, etc.). In certain embodiments, the presently disclosed methods include a selection of CD3+ immunoresponsive cells. In certain embodiments, the selection is a positive selection. During positive selection, cells bind to a specific reagent and are retained for further use. In certain embodiments, the positive selection provides an enrichment of a cell expressing a particular marker. In certain embodiments, the positive selection does not result in 100% enrichment of a particular cell. For example, but without any limitation, positive selection for CD4+ cells can increase the number of CD4+ cells but does not need to result in a complete absence of non-CD4+ cells. In certain embodiments, the selection includes contacting a sample (e.g., one described in Section 5.2.1. above) with a binding reagent (e.g., a CD3⁺ monospecific reagent, e.g., an anti-CD3 antibody). In certain embodiments, the binding reagent is a CD3⁺ monospecific reagent. In certain embodiments, the selection does not activate the immunoresponsive cell.

### 5.2.2.1. Binding Reagent

In certain embodiments, the sample is contacted with a binding reagent.

In certain embodiments, the binding reagent is directly or indirectly bound to a solid support (e.g., matrix, bead, etc.) to allow for the separation of cells for positive and/or negative selection. In certain embodiments, the solid support is a magnetic particle. In certain embodiments, the magnetic particle can be a magnetic bead, a magnetic microbead, a paramagnetic bead, or a paramagnetic microbead. Non-limiting examples of magnetic particles encompassed by the presently disclosed subject matter include Dynabeads® (Life Technologies, Carlsbad, CA), MACS® beads (Miltenyi Biotec, San Diego, CA), or Streptamer® bead reagents (IBA, Germany).

In certain embodiments, the binding reagent (e.g., a CD3⁺ monospecific reagent) binds specifically to a surface marker of the desired immunoresponsive cell. Non-limiting examples of binding reagents include antibodies, binding proteins, aptamers, etc. In certain embodiments, the binding reagent is a CD3⁺ monospecific reagent.

In certain embodiments, the binding reagent is an antibody (e.g., a CD3⁺ monospecific reagent, e.g., an anti-CD3 antibody). In certain embodiments, the antibody is an anti-CD3 antibody. In certain embodiments, the anti-CD3 antibody is bound directly to a microbead. In certain embodiments, the anti-CD3 antibody is bound indirectly to a magnetic particle (e.g., using streptavidin/biotin system).

Anti-CD3 antibodies have traditionally been barred from methods for preparing adoptive cell therapies because of their potent immunosuppressive effect. It was generally assumed that anti-CD3 antibodies could induce tolerance mechanisms through the induction of naive T cell unresponsiveness, often referred to as anergy (see Andris et al., J Immunol. 2004 Sep. 1;173 (5): 3201-8). The inventors of the presently disclosed subject matter surprisingly discovered that anti-CD3 antibodies can be used for positive selection of CD3⁺ immunoresponsive cells without induction of anergy. In certain embodiments, the anti-CD3 antibody binds to an epitope located on the human CD3ε chain. In certain embodiments, the anti-CD3 antibody comprises a dissociation constant (K_{D}) from about 10⁻⁵ M to about 10⁻¹¹ M, from about 10⁻⁶ M to about 10⁻¹¹ M, from about 10⁻⁷ M to about 10⁻¹¹ M, from about 10⁻⁸ M to about 10⁻¹¹ M, from about 10⁻⁹ M to about 10⁻¹¹ M, from about 10⁻¹⁰ M to about 10⁻¹¹ M, from about 10⁻⁵ M to about 10⁻¹⁰ M from about 10⁻⁵ M to about 10⁻⁹ M, from about 10⁻⁵ M to about 10⁻⁸ M, from about 10⁻⁵ M to about 10⁻⁷ M, or from about 10⁻⁵ M to about 10⁻⁶ M. In certain embodiments, the anti-CD3 antibody comprises a dissociation constant (K_{D}) from about 10⁻⁹ M to about 10⁻¹¹ M. In certain embodiments, the anti-CD3 antibody comprises a dissociation constant (K_{D}) of about 10⁻¹⁰ M to about 10⁻¹¹ M. In certain embodiments, the anti-CD3 antibody comprises a dissociation constant (K_{D}) from about 10⁻¹⁰ M to about 1.2×10⁻¹⁰ M. In certain embodiments, the anti-CD3 antibody comprises a dissociation constant (K_{D}) of about 1.06×10⁻¹⁰ M. In certain embodiments, the anti-CD3 antibody comprises a dissociation constant (K_{D}) of about 1.24×10⁻¹⁰ M.

In certain embodiments, the anti-CD3 antibody comprises a heavy chain variable region (V_{H}) and a light chain variable region (V_{L}). In certain embodiments, the heavy chain variable region (V_{H}) comprises or consists of an amino acid sequence that is at least about 85%, about 90%, about 95%, about 96%, about 97%, about 98%, about 99% or about 100% identical or homologous to the amino acid sequence set forth in SEQ ID NO: 55. In certain embodiments, the heavy chain variable region (V_{H}) comprises or consists of the amino acid sequence set forth in SEQ ID NO: 55.

In certain embodiments, the light chain variable region (V_{L}) comprises or consists of an amino acid sequence that is at least about 85%, about 90%, about 95%, about 96%, about 97%, about 98%, about 99% or about 100% identical or homologous to the amino acid sequence set forth in SEQ ID NO: 56. In certain embodiments, the light chain variable region (V_{L}) comprises or consists of the amino acid sequence set forth in SEQ ID NO: 56.

In certain embodiments, the anti-CD3 antibody comprises a heavy chain variable region (V_{H}) comprising or consisting of an amino acid sequence that is at least about 85%, about 90%, about 95%, about 96%, about 97%, about 98%, about 99% or about 100% identical or homologous to the amino acid sequence set forth in SEQ ID NO: 55 and a light chain variable region (V_{L}) comprising or consisting of an amino acid sequence that is at least about 85%, about 90%, about 95%, about 96%, about 97%, about 98%, about 99% or about 100% identical or homologous to the amino acid sequence set forth in SEQ ID NO: 56.

In certain embodiments, the anti-CD3 antibody comprises a heavy chain variable region (V_{H}) comprising or consisting of the amino acid sequence set forth in SEQ ID NO: 55 and a light chain variable region (V_{L}) comprising or consisting of the amino acid sequence set forth in SEQ ID NO: 56. SEQ ID NO: 55 and SEQ ID NO: 56 are provided below:

[SEQ ID NO: 55]
QVQLQQSGAELARPGASVKMSCKASGYTFTRYTMHWVKQRPGQGL
EWIGYINPSRGYTNYNQKEKDKATLTTDKSSSTAYMQLSSLTSED
SAVYYCARYYDDHYCLDYWGQGTTLTVSS
[SEQ ID NO: 56]
QIVLTQSPAIMSASPGEKVTMTCSASSSVSYMNWYQQKSGTSPKR
WIYDTSKLASGVPAHERGSGSGTSYSLTISGMEAEDAATYYCQQW
SSNPFTFGSGTKLEIN

In certain embodiments, the CD3⁺ monospecific reagent does not activate the immunoresponsive cell. In certain embodiments, the anti-CD3 antibody does not activate the immunoresponsive cell.

In certain embodiments, the magnetic particle has a diameter from about 10 nm to about 50 μm. In certain embodiments, the magnetic particle has a diameter from about 10 nm to about 100 nm, from about 0.1 μm to about 1 μm, from about 1 μm to about 50 μm. In certain embodiments, the magnetic particle has a diameter from about 10 nm to about 100 nm, from about 10 nm to about 90 nm, from about 10 nm to about 80 nm, from about 10 nm to about 70 nm, from about 10 nm to about 60 nm, from about 10 nm to about 50 nm, from about 10 nm to about 40 nm, from about 10 nm to about 30 nm, from about 10 nm to about 20 nm, from about 20 nm to about 60, from about 30 nm to about 60 nm, from about 40 nm to about 60 nm, from about 20 nm to about 100 nm, from about 30 nm to about 100 nm, from about 40 nm to about 100 nm, from about 50 nm to about 100 nm, from about 60 nm to about 100 nm, from about 70 nm to about 100 nm, from about 80 nm to about 100 nm, or from about 90 nm to about 100 nm. In certain embodiments, the magnetic particle has a diameter of about 50 nm.

In certain embodiments, the magnetic particle comprises an iron carbohydrate complex. Non-limiting examples of iron carbohydrate complexes encompassed by the presently disclosed subject matter include iron carboxymaltose, iron polyglucose sorbitol carboxymethyl ether complex, iron mannitol complex, iron dextran, iron hydrogenated dextran, iron oxidized dextran, iron carboxyalkylated reduced oligo- and polysaccharides, iron sucrose, iron gluconate, iron dextrin, iron hydrogenated dextrin, iron oxidized dextrin, iron polymaltose, iron hydrogenated polymaltose, iron oligomaltose, hydrogenated iron oligomaltose, iron polyisomaltose, iron hydrogenated polyisomaltose, iron hydrogenated oligosaccharides (e.g., iron hydrogenated oligoisomaltose), iron hydroxyethyl starch, iron sorbitol, or iron dextran glucoheptonic acid. In certain embodiments, the magnetic particle comprises iron dextran.

In certain embodiments, the sample is contacted with the binding reagent (e.g., a CD3⁺ monospecific reagent) at a cell density between about 1×10⁶ cells/ml to about 100×10⁷ cells/ml. In certain embodiments, the cell density is between about 1× 10⁶ cells/ml to about 10×10⁷ cells/ml, between about 1×10⁶ cells/ml to about 1× 10⁷ cells/ml, between about 1×10⁶ cells/ml to about 100×10⁶ cells/ml, between about 1× 10⁶ cells/ml to about 10× 10⁶ cells/ml, between about 10×10⁶ cells/ml to about 100× 10⁷ cells/ml, between about 100×10⁶ cells/ml to about 100× 10⁷ cells/ml, between about 1×10⁷ cells/ml to about 100×10⁷ cells/ml, or between about 10×10⁷ cells/ml to about 100×10⁷ cells/ml. In certain embodiments, the sample is contacted with the binding reagent at a cell density of about 100×10⁶ cells/ml.

In certain embodiments, the binding reagent is Miltenyi™ CD3 MicroBeads. In certain embodiments, the binding reagent is CliniMACS® CD3 GMP MicroBeads.

### 5.2.2.2. Separation of Cells

In certain embodiments, the presently disclosed subject matter comprises the separation of a desired immunoresponsive cell (e.g., a CD3⁺ immunoresponsive cell) using immuno-magnetic or affinity-magnetic separation techniques.

In certain embodiments, the separation is carried out by placing the sample including the binding reagent (e.g., CD3⁺ monospecific reagent) under a magnetic field. In certain embodiments, the separation is carried out via magnetic-activated cell sorting (MACS) (Miltenyi Biotech, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. Initially, the cells attached to magnetic particles are held in place while the unattached species are eluted. After this first elution step is completed, the cells that were trapped in the magnetic field are eluted and recovered.

In certain embodiments, additional separations can be carried out. For example, the positively selected cells can be subjected to another separation using a different binding reagent. For example, but without any limitation, a specific subpopulation of immunoresponsive cells (e.g., T cells expressing CD28⁺, CD62L⁺, CCR7⁺, CD27⁺, CD127⁺, CD4⁺, CD8⁺, CD45RA⁺, and/or CD45RO+) can be obtained by additional separation using specific binding reagents.

In certain embodiments, the presently disclosed subject matter comprises the separation of a desired immunoresponsive cell (e.g., a CD3⁺ immunoresponsive cell) using immunoaffinity separation techniques (e.g., immunoaffinity chromatography). In certain embodiments, the immunoaffinity separation technique comprises a chromatography column.

In certain embodiments, the chromatography column comprises a stationary phase (e.g., matrix). In certain embodiments, the stationary phase comprises a polymeric resin, a metal oxide, or a metalloid oxide. In certain embodiments, the stationary phase comprises a non-magnetic material or non-magnetizable material. In certain embodiments, the stationary phase comprises derivatized silica. In certain embodiments, the stationary phase comprises a crosslinked gel. In certain embodiments, the stationary phase comprises an agarose gel. Non-limiting examples of materials used in stationary phases disclosed herein include Superflow™ agarose, Superflow™ Sepharose®, Sephadex®, Superdex®, or Sephacryl®. In certain embodiments, the stationary phase comprises a synthetic polymer (e.g., polyacrylamide, styrene-divinylbenzene gel, etc.). In certain embodiments, the stationary phase is packed into a column.

In certain embodiments, the stationary phase comprises a binding reagent (e.g., a CD3⁺ monospecific reagent disclosed above). In certain embodiments, the binding reagent (e.g., a CD3⁺ monospecific reagent) is an antibody (e.g., an anti-CD3 antibody). In certain embodiments, the antibody is an anti-CD3 antibody. In certain embodiments, the anti-CD3 antibody does not activate the immunoresponsive cell.

In certain embodiments, the chromatography column can be used for selection methods as described herein. For example, the selection methods can include contacting the stationary phase with the sample.

In certain embodiments, multiple chromatography columns can be used. For example, but without any limitation, two chromatography columns can be operably connected, wherein a first column including an anti-CD3 antibody is coupled with a second column including an anti-CD4 antibody. Additional information regarding the use of multiple chromatography columns can be found in Wingfieldet al., Current protocols in protein science 80, no. 1 (2015): 6-1, the content of which is incorporated by reference in its entirety.

### 5.2.2.3. Compositions Enriched for CD3+Immunoresponsive Cells

Upon separation of the cells, a composition (e.g., eluate) enriched for the desired cell type (e.g., a CD3⁺ immunoresponsive cell) is obtained. Using a single positive selection process (e.g., using a presently disclosed CD3⁺ monospecific reagent as described above), the presently disclosed subject matter provides a composition enriched for CD3⁺, CD4⁺, and/or CD8⁺ immunoresponsive cells.

In certain embodiments, the composition is enriched for CD3⁺ immunoresponsive cells (e.g., compared to the sample). In certain embodiments, the composition comprises at least about 80%, at least about 85%, at least about 90%, at least about 95%, at least about 96%, at least about 97%, at least about 98%, at least about 99%, or about 100% more CD3⁺ immunoresponsive cells compared to the sample. In certain embodiments, the composition comprises at least about 90% more CD3⁺ immunoresponsive cells compared to the sample. In certain embodiments, the composition comprises at least about 60%, at least about 65%, at least about 70%, at least about 75%, at least about 80%, at least about 85%, at least about 90%, at least about 95%, at least about 96%, at least about 97%, at least about 98%, at least about 99%, or about 100% of CD3+ immunoresponsive cells over the total number of cells. In certain embodiments, the composition comprises at least about 90% of CD3⁺ immunoresponsive cells over the total number of cells.

In certain embodiments, the composition is enriched for CD4⁺ immunoresponsive cells (e.g., compared to the sample). In certain embodiments, the composition comprises at least about 30%, at least about 45%, at least about 50%, at least about 60%, at least about 70%, at least about 80%, at least about 90%, at least about 95% more CD4⁺ immunoresponsive cells compared to the sample. In certain embodiments, the composition comprises at least about 40% more CD4⁺ immunoresponsive cells compared to the sample. In certain embodiments, the composition comprises at least about 30%, at least about 45%, at least about 50%, at least about 60%, at least about 70%, at least about 80%, at least about 90%, at least about 95% of CD4⁺ immunoresponsive cells over the total number of cells. In certain embodiments, the composition comprises at least about 30% of CD4⁺ immunoresponsive cells over the total number of cells.

In certain embodiments, the composition is enriched for CD8⁺ immunoresponsive cells (e.g., compared to the sample). In certain embodiments, the composition comprises at least about 30%, at least about 45%, at least about 50%, at least about 60%, at least about 70%, at least about 80%, at least about 90%, at least about 95% more CD8⁺ immunoresponsive cells compared to the sample. In certain embodiments, the composition comprises at least about 40% more CD8⁺ immunoresponsive cells compared to the sample. In certain embodiments, the composition comprises at least about 30%, at least about 45%, at least about 50%, at least about 60%, at least about 70%, at least about 80%, at least about 90%, at least about 95% of CD8⁺ immunoresponsive cells over the total number of cells. In certain embodiments, the composition comprises at least about 30% of CD8⁺ immunoresponsive cells over the total number of cells.

In certain embodiments, the composition has fewer CD14⁺ immunoresponsive cells (e.g., compared to the sample). In certain embodiments, the composition comprises at least about 80%, at least about 85%, at least about 90%, at least about 95%, at least about 96%, at least about 97%, at least about 98%, at least about 99%, or about 100% less CD14⁺ immunoresponsive cells compared to the sample. In certain embodiments, the composition comprises at least about 80% fewer CD14⁺ immunoresponsive cells compared to the sample. In certain embodiments, the composition comprises at least about 1%, at least about 5%, at least about 10%, at least about 15%, or at least about 20% of CD14⁺ immunoresponsive cells over the total number of cells. In certain embodiments, the composition comprises at least about 5% of CD14⁺ immunoresponsive cells over the total number of cells.

In certain embodiments, the composition has fewer CD33⁺ immunoresponsive cells (e.g., compared to the sample). In certain embodiments, the composition comprises at least about 80%, at least about 85%, at least about 90%, at least about 95%, at least about 96%, at least about 97%, at least about 98%, at least about 99%, or about 100% less CD33⁺ immunoresponsive cells compared to the sample. In certain embodiments, the composition comprises at least about 80% fewer CD33⁺ immunoresponsive cells compared to the sample. In certain embodiments, the composition comprises at least about 1%, at least about 5%, at least about 10%, at least about 15%, or at least about 20% of CD33⁺ immunoresponsive cells over the total number of cells. In certain embodiments, the composition comprises at least about 5% of CD33⁺ immunoresponsive cells over the total number of cells.

In certain embodiments, the composition has fewer CD34⁺ immunoresponsive cells (e.g., compared to the sample). In certain embodiments, the composition comprises at least about 80%, at least about 85%, at least about 90%, at least about 95%, at least about 96%, at least about 97%, at least about 98%, at least about 99%, or about 100% less CD34⁺ immunoresponsive cells compared to the sample. In certain embodiments, the composition comprises at least about 80% fewer CD34⁺ immunoresponsive cells compared to the sample. In certain embodiments, the composition comprises at least about 1%, at least about 5%, at least about 10%, at least about 15%, or at least about 20% of CD34⁺ immunoresponsive cells over the total number of cells. In certain embodiments, the composition comprises at least about 5% of CD34⁺ immunoresponsive cells over the total number of cells.

In certain embodiments, the composition is enriched for CD3⁺, CD4⁺, and CD8⁺ immunoresponsive cells (e.g., compared to the sample). In certain embodiments, the composition has fewer CD14⁺, CD33⁺, and CD34⁺ immunoresponsive cells (e.g., compared to the sample). In certain embodiments, the composition is enriched for CD3⁺, CD4⁺, and CD8⁺ immunoresponsive cells and has fewer CD14⁺, CD33⁺, and CD34⁺ immunoresponsive cells (e.g., compared to the sample).

### 5.2.2.4. Activation and Expansion

To achieve sufficient therapeutic doses for adoptive cell therapies, immunoresponsive cell compositions (e.g., one described in Section 5.2.2.3) are subject to one or more rounds of stimulation, activation, and/or expansion.

In certain embodiments, the presently disclosed subject matter provides methods including activating and expanding immunoresponsive cells. Activation and expansion of immunoresponsive cells (e.g., T cells) require a first signal through the T cell TCR/CD3 complex or by stimulation of the CD2 surface protein, and a second signal (e.g., a costimulatory signal) by a co-stimulatory molecule (e.g., CD28 agonist).

In certain embodiments, the first signal can be provided by contacting the immunoresponsive cell with a CD3 binding reagent. Non-limiting examples of CD3 binding reagents capable of activating an immunoresponsive cell include the OKT3 antibody, the G19-4 antibody, the BC3 antibody, and the 64.1 antibody.

In certain embodiments, the first signal can be provided by contacting the immunoresponsive cell with a CD2 binding reagent. Non-limiting examples of CD2 binding reagents capable of activating an immunoresponsive cell include the T11.3 antibody, the T11.1 antibody, the T11.2 antibody, the 9.6 antibody, and the 9-1 antibody.

In addition to the first signal, activation, and expansion of immunoresponsive cells requires a second signal (e.g., a costimulatory signal). In certain embodiments, the second signal can be provided by contacting the immunoresponsive cell with a CD28 binding reagent. Non-limiting examples of CD28 binding reagents include natural ligands for CD28 (e.g., a member of the B7 family of proteins like B7-1 (CD80) and B7-2 (CD86), and anti-CD28 monoclonal antibody (e.g., monoclonal antibodies 9.3, B-T3, and XR-CD28).

In certain embodiments, the first signal and the second signal are provided by the same compound. For example, but without any limitation, a compound can include a first binding reagent delivering the first signal (e.g., CD3 binding reagent) and a second binding reagent delivering the second signal (e.g., CD28 binding reagent). In certain embodiments, the first signal and the second signal are provided by a magnetic particle including a first binding reagent delivering the first signal (e.g., CD3 binding reagent) and a second binding reagent delivering the second signal (e.g., CD28 binding reagent). In certain embodiments, the first signal and the second signal are provided by a magnetic particle including an anti-CD3 antibody and an anti-CD28 antibody. For example, but without any limitation, the first signal and the second signal are provided by Dynabeads™ Human T-Activator CD3/CD28, ProMab Biotechnologies D28/CD3 PM-T-Cell Activation/Expansion Beads, or T Cell TransAct™.

In certain embodiments, the presently disclosed methods including activation and expansion of immunoresponsive cells further include culturing the immunoresponsive cells to achieve a desired number of cells (e.g., 100×10⁷ cells). In certain embodiments, culturing can include the use of cytokines and growth factors to induce proliferation and improve viability. For example, but without any limitation, cytokines and growth factors to induce proliferation and improve viability include interleukin-2 (IL-2), insulin, IFN-γ, IL-4, IL-7, IL-21, GM-CSF, IL-10, IL-12, IL-15, TGFβ3, and TNF-α.

In certain embodiments, immunoresponsive cell compositions (e.g., described in Section 5.2.2.3) are contacted with a stimulatory agent and costimulatory agent (e.g., T Cell TransAct™) in a culture medium with cytokines and growth factors (e.g., IL-2, IL-7, and/or IL-15).

Alternatively or additionally, the first signal and the second signal can be provided by artificial antigen-presenting cells displaying on their surfaces CD3 binding reagent, CD2 binding reagent, and/or CD28 binding reagent. Additional information on artificial antigen-presenting cells can be found in Neal et al., Journal of immunology research and therapy 2, no. 1 (2017): 68, the content of which is incorporated by reference in its entirety.

The immunoresponsive cells can be modified to express an antigen-recognizing receptor. In certain embodiments, the immunoresponsive cells are modified before activation and expansion. In certain embodiments, the immunoresponsive cells are modified after activation and expansion. In certain embodiments, the immunoresponsive cells are modified after activation and before expansion.

### 5.2.2.5. Vectors and Delivery of the Antigen-Recognizing Receptor

The presently disclosed subject matter provides nucleic acid compositions comprising a polynucleotide encoding an antigen-recognizing receptor disclosed herein. Also provided are cells comprising such nucleic acid compositions. In certain embodiments, the nucleic acid composition further comprises a promoter that is operably linked to the antigen-recognizing receptor.

In addition, the presently disclosed subject matter provides nucleic acid compositions comprising a polynucleotide encoding a fusion polypeptide disclosed herein. Also provided are cells comprising such nucleic acid compositions. In certain embodiments, the nucleic acid composition further comprises a promoter that is operably linked to the fusion polypeptide. In certain embodiments, the nucleic acid composition further comprises a promoter that is operably linked to an antigen-recognizing receptor. In certain embodiments, one or both of the first and second promoters are endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiments, the inducible promoter is selected from a NFAT transcriptional response element (TRE) promoter, a CD69 promoter, a CD25 promoter, and an IL-2 promoter.

In certain embodiments, the nucleic acid composition is a vector. In certain embodiments, the vector is a retroviral vector (e.g., a gamma-retroviral vector or a lentiviral vector). In certain embodiments, the vector is viral vectors selected from the group consisting of adenoviral vectors, adena-associated viral vectors, vaccinia viruses, bovine papilloma viruses, and herpes viruses (e.g., such as Epstein-Barr Virus).

Additionally, the nucleic acid compositions can be administered to introduced and/or delivered into cells by art-known methods or as described herein. Genetic modification of a cell (e.g., a T cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, the first polynucleotide and the second polynucleotide can be cloned into a retroviral vector and expression can be driven from its endogenous promoter, from the retroviral long terminal repeat, or from a promoter-specific for a target cell type of interest. Non-viral vectors may be used as well.

The first polynucleotide and the second polynucleotide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114, or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107: 77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the genetic modification of a cell. For example, a nucleic acid molecule can be delivered into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but are not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the antigen-recognizing receptor is delivered to the cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the antigen-recognizing receptor is delivered to the cell by a non-viral method. Any targeted genome editing methods can also be used to deliver the antigen-recognizing receptor to the cell. In certain embodiments, the antigen-recognizing receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the antigen-recognizing receptor to the cell.

In certain embodiments, a CRISPR system is used to deliver the antigen-recognizing receptor to the cell. Clustered regularly-interspaced short palindromic repeats (CRISPR) system is a genome-editing tool discovered in prokaryotic cells. When utilized for genome editing, the system includes Cas9 (a protein able to modify DNA utilizing crRNA as its guide), CRISPR RNA (crRNA, which contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying the CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. In certain embodiments, the CRISPR system comprises base editors. In certain embodiments, the CRISPR system comprises transposases/recombinases. In certain embodiments, the CRISPR system comprises prime editors. In certain embodiments, the CRISPR system comprises an epigenetic modulator. In certain embodiments, the CRISPR system comprises is a CRISPRoff system. Additional details on the CRISPR systems of the presently disclosed subject matter can be found in Anzalone et al., Nature biotechnology 38.7 (2020): 824-844 and in Nuñez et al., Cell 184.9 (2021): 2503-2519, the contents of each of which are incorporated by reference in their entireties.

In certain embodiments, zinc-finger nucleases are used to deliver the antigen-recognizing receptor to the cell. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, a TALEN system is used to deliver the antigen-recognizing receptor to the cell. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor la enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In certain embodiments, upon delivery of the antigen-recognizing receptor to the cell, the antigen-recognizing receptor is integrated at a locus within the genome of the T cell, e.g., a TRAC locus, a TRBC locus, a TRDC locus, or a TRGC locus. In certain embodiments, the locus is a TRAC locus. In certain embodiments, the expression of the antigen-recognizing receptor is under the control of an endogenous promoter. Non-limiting examples of endogenous promoters include an endogenous TRAC promoter, an endogenous TRBC promoter, an endogenous TRDC promoter, and an endogenous TRGC promoter. In certain embodiments, the endogenous promoter is an endogenous TRAC promoter.

### 5.2.2.6. Storage of Cells

The presently disclosed methods also include cryopreservation of the immunoresponsive cells. In certain embodiments, the immunoresponsive cells are viable upon thawing. As used herein, the term “cryopreservation” refers to a process that preserves organelles, cells, tissues, or any other biological constructs by cooling the samples to very low temperatures. In certain embodiments, the cryopreservation includes cooling to sub-zero temperatures (e.g., −196° C.). In certain embodiments, the cryopreservation includes the use of cryoprotective agents. Non-limiting examples of cryoprotective agents include dimethyl sulfoxide (DMSO) glycerol, polyvinylpyrrolidone, and polyethylene glycol. In certain embodiments, the cryopreservation comprises controlled rate cooling. In certain embodiments, the controlled rate cooling is from about 1° C. to about 3° C./minute. In certain embodiments, the controlled rate cooling stops once a temperature of −80° C. has been reached.

### 5.3. Cells

The presently disclosed subject matter provides cells prepared through the methods disclosed in Section 5.2.

In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell). In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and B heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G.A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the cell (e.g., T cell) is autologous. In certain embodiments, the cell (e.g., T cell) is non-autologous. In certain embodiments, the cell (e.g., T cell) is allogeneic. In certain embodiments, the cell (e.g., T cell) is derived in vitro from an engineered progenitor or stem cell.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

### 5.3.1. Antigen-Recognizing Receptors

The presently disclosed subject matter provides cells (e.g., prepared through the methods disclosed in Section 5.2) including an antigen-recognizing receptor. In certain embodiments, the antigen-recognizing receptor targets an antigen. In certain embodiments, the antigen can be a tumor antigen or a pathogen antigen. In certain embodiments, the antigen-recognizing receptor is a chimeric receptor. In certain embodiments, the chimeric receptor is a chimeric antigen receptor (CAR). In certain embodiments, the antigen-recognizing receptor is a TCR like fusion molecule. In certain embodiments, the antigen-recognizing receptor is a T Cell Receptor (TCR). In certain embodiments, the antigen-recognizing receptor is a chimeric costimulatory receptor (CCR).

### 5.3.1.1. Antigen

In certain embodiments, the antigen is a tumor antigen. Any tumor antigen (antigenic peptide) can be used in the tumor-related embodiments described herein. Sources of antigen include, but are not limited to, cancer proteins. The antigen can be expressed as a peptide or as an intact protein or a portion thereof. The intact protein or portion thereof can be native or mutagenized. Non-limiting examples of tumor antigens include CD19, CD70, IL1RAP, ABCG2, AChR, ACKR6, ADAMTS13, ADGRE2, ADGRE2 (EMR2), ADORA3, ADRAID, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLECIA, CLL1, CNIH2, COL15A1, COLEC12, CPM, CR1, CX3CR1, CXCR4, CYP4F11, DAGLB, DARC, DFNB31, DGKI, EGFIR, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGRIA, FKBPIB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL1ORB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LICAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNNID, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the antigen is CD19. In certain embodiments, the antigen is expressed on an acute myeloid leukemia (AML) tissue. In certain embodiments, the antigen is expressed on an acute myeloid leukemia (AML) hematopoietic stem/progenitor cell (HSPC) and/or a leukemia stem cell (LSC). In certain embodiments, the AML HSPC expresses CD34. In certain embodiments, the antigen is expressed in a malignant hematopoietic stem cell and/or a malignant hematopoietic progenitor cell. In certain embodiments, the antigen is not expressed or expressed at a non-detectable level in a non-malignant hematopoietic stem cell and/or a non-malignant hematopoietic progenitor cell. In certain embodiments, the antigen is selected from the group consisting of CD70, IL1RAP, CD19, CD33, CLEC12A, ADGRE2, CD123, and combinations thereof.

In certain embodiments, the antigen is a pathogen antigen. Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g. M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, corynebacterium sp., Erysipelothrix rhusiopathiae, Clostridium spp., Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 5.3.1.2. Chimeric Receptors

In certain embodiments, the antigen-recognizing receptor is a chimeric receptor. In certain embodiments, the chimeric receptor is a chimeric antigen receptor (CAR).

### 5.3.1.2.1 Chimeric Antigen Receptors (CARs)

CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARS. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs include a signaling domain of a co-stimulatory molecule (e.g., CD28, 4-1BB, ICOS, OX40, CD27, CD40,NKG2D, DAP-10, CD2, CD150, CD226) to the intracellular signaling domain of the CAR to provide co-stimulation signals to the cell (e.g., T cell or NK cell). “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ).

In certain embodiments, the antigen-recognizing receptor is a CAR comprising an extracellular antigen-binding domain that binds to the antigen, and an intracellular signaling domain. In certain embodiments, the CAR further comprises a transmembrane domain. In certain embodiments, the CAR further comprises a hinger/spacer region.

In certain embodiments, the extracellular antigen-binding domain of the CAR (for example, an scFv) binds to the first antigen with a dissociation constant (K_{D}) of about 5×10⁻⁷ M or less, about 1×10⁻⁷ M or less, about 5×10⁻⁸ M or less, about 1×10⁻⁸ M or less, about 5×10⁻⁹ M or less, or about 1×10⁻⁹M or less, or about 1×10⁻¹⁰ M or less.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

The extracellular antigen-binding domain can comprise or be an scFv, a Fab (which is optionally crosslinked), or a F(ab)₂. In certain embodiments, any of the foregoing molecules may be comprised in a fusion protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

In certain embodiments, the antigen-recognizing receptor is a CAR that comprises a transmembrane domain. Different transmembrane domains result in different receptor stability. After antigen recognition, receptors cluster and a signal are transmitted to the cell. In accordance with the presently disclosed subject matter, the transmembrane domain of the antigen-recognizing receptor can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain of the CAR comprises a CD28 polypeptide (e.g., the transmembrane domain of CD28 or a portion thereof). In certain embodiments, the transmembrane domain of the CAR comprises a transmembrane domain of human CD28 or a portion thereof. In certain embodiments, the CD28 polypeptide comprises or consists of an amino acid sequence that is a consecutive portion of the amino acid sequence having a NCBI Reference No: NP_006130 (SEQ ID NO: 7), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 25. In certain embodiments, the transmembrane domain of the CAR comprises a CD28 polypeptide that comprises or consists of amino acids 153 to 179 of SEQ ID NO: 7. SEQ ID NO: 7 is provided below.

[SEQ ID NO: 7]
MLRLLLALNLFPSIQVTGNKILVKQSPMLVAYDNAVNLSCKYSYN
LESREFRASLHKGLDSAVEVCVVYGNYSQQLQVYSKTGENCDGKL
GNESVTFYLQNLYVNQTDIYFCKIEVMYPPPYLDNEKSNGTIIHV
KGKHLCPSPLFPGPSKPFWVLVVVGGVLACYSLLVTVAFIIFWVR
SKRSRLLHSDYMNMTPRRPGPTRKHYQPYAPPRDFAAYRS

In certain embodiments, the antigen-recognizing receptor is a CAR that further comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3 (polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 7), a portion of a CD8 polypeptide, or a synthetic spacer sequence.

In certain embodiments, the antigen-recognizing receptor is a CAR that further comprises a hinge/spacer region comprising a native or modified hinge region of a CD28 polypeptide. In certain embodiments, the hinge/spacer region of the first antigen-recognizing receptor (e.g., a CAR) comprises a CD28 polypeptide comprising or consisting of amino acids 114 to 152 of SEQ ID NO: 7.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3 polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8a polypeptide and the transmembrane domain comprises a CD8a polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

In certain embodiments, the antigen-recognizing receptor is a CAR that comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T-cell) after antigen is bound. The intracellular signaling domain of the CD33-chain is the primary transmitter of signals from endogenous TCRs.

In certain embodiments, the intracellular signaling domain of the CAR comprises a native CD32. In certain embodiments, the native CD3ζ comprises or consists of an amino acid sequence that is at least about 85%, about 90%, about 95%, about 96%, about 97%, about 98%, about 99% or about 100% identical or homologous to the amino acid sequence having a NCBI Reference No: NP_932170 (SEQ ID NO: 8) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 8, which is at least about 20, or at least about 30, or at least about 40, or at least about 50, and up to about 164 amino acids in length. In certain embodiments, the native CD3ζ comprises or consists of the amino acid sequence of amino acids 1 to 164, 1 to 50, 50 to 100, 52 to 164, 100 to 150, or 150 to 164 of SEQ ID NO: 8. In certain embodiments, the intracellular signaling domain of the CAR comprises a native CD3ζ comprising or consisting of the amino acid sequence of amino acids 52 to 164 of SEQ ID NO: 8. SEQ ID NO: 8 is provided below:

[SEQ ID NO: 8]
MKWKALFTAAILQAQLPITEAQSFGLLDPKLCYLLDGILFIYGVI
LTALFLRVKFSRSADAPAYQQGQNQLYNELNLGRREEYDVLDKRR
GRDPEMGGKPQRRKNPQEGLYNELQKDKMAEAYSEIGMKGERRRG
KGHDGLYQGLSTATKDTYDALHMQALPPR

In certain embodiments, the native CD3ζ comprises or consists of an amino acid sequence that is at least about 85%, about 90%, about 95%, about 96%, about 97%, about 98%, about 99% or about 100% identical or homologous to the amino acid sequence set forth in SEQ ID NO: 9. SEQ ID NO: 9 is provided below:

[SEQ ID NO: 9]
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 10.

[SEQ ID NO: 10]
QNQLYNELNLGRREEYDVLDKR

An exemplary nucleic acid sequence encoding the amino acid sequence of SEQ ID NO: 10 is set forth in SEQ ID NO: 11, which is provided below.

[SEQ ID NO: 11]
CAGAACCAGCTCTATAACGAGCTCAATCTAGGACGAAGAGAGGAG
TACGATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 12, which is provided below.

[SEQ ID NO: 12]
QNQLENELNLGRREEFDVLDKR

An exemplary nucleic acid sequence encoding the amino acid sequence of SEQ ID NO: 12 is set forth in SEQ ID NO: 13, which is provided below.

[SEQ ID NO: 13]
CAGAACCAGCTCTTTAACGAGCTCAATCTAGGACGAAGAGAGGAG
TTCGATGTTTTGGACAAGAGA

In certain embodiments, the modified CD3C polypeptide comprises a native ITAM2. In certain embodiments, the native ITAM2 comprises or consists of the amino acid sequence set forth in SEQ ID NO: 14, which is provided below.

[SEQ ID NO: 14]
QEGLYNELQKDKMAEAYSEIGMK

An exemplary nucleic acid sequence encoding the amino acid sequence of SEQ ID NO: 14 is set forth in SEQ ID NO: 15, which is provided below.

[SEQ ID NO: 15]
CAGGAAGGCCTGTACAATGAACTGCAGAAAGATAAGATGGCGGAG
GCCTACAGTGAGATTGGGATGAAA

In certain embodiments, the modified CD3 polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 16, which is provided below.

[SEQ ID NO: 16]
QEGLFNELQKDKMAEAFSEIGMK

An exemplary nucleic acid sequence encoding the amino acid sequence of SEQ ID NO: 16 is set forth in SEQ ID NO: 17, which is provided below.

[SEQ ID NO: 17]
CAGGAAGGCCTGTTCAATGAACTGCAGAAAGATAAGATGGCGGAGGCCTT
CAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 18, which is provided below.

[SEQ ID NO: 18]
HDGLYQGLSTATKDTYDALHMQ

An exemplary nucleic acid sequence encoding the amino acid sequence of SEQ ID NO: 18 is set forth in SEQ ID NO: 19, which is provided below.

[SEQ ID NO: 19]
CACGATGGCCTTTACCAGGGTCTCAGTACAGCCACCAAGGACACCTACGA
CGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 20, which is provided below.

[SEQ ID NO: 20]
HDGLFQGLSTATKDTFDALHMQ

An exemplary nucleic acid sequence encoding the amino acid sequence of SEQ ID NO: 20 is set forth in SEQ ID NO: 21, which is provided below.

[SEQ ID NO: 21]
CACGATGGCCTTTTCCAGGGGCTCAGTACAGCCACCAAGGACACCTTCGA
CGCCCTTCACATGCAG

Various modified CD3ζ polypeptides and CARs comprising modified CD3% polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 10, an ITAM2 variant consisting of the amino acid sequence set forth in SEQ ID NO: 16, and an ITAM3 variant consisting of the amino acid sequence set forth in SEQ ID NO: 20. In certain embodiments, the CAR is designated as “1XX”. In certain embodiments, the modified CD3ζ polypeptide comprises or consists of the amino acid sequence set forth in SEQ ID NO: 22. SEQ ID NO: 22 is provided below:

[SEQ ID NO: 22]
RVKFSRSADAPAYQQGQNQLYNELNLGRREEYDVLDKRRGRDPEMGGKPR
RKNPQEGLFNELQKDKMAEAFSEIGMKGERRRGKGHDGLFQGLSTATKDT
FDALHMQALPPR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 22 or a fragment thereof, and/or may optionally comprise up to one or up to two or up to three conservative amino acid substitutions.

An exemplary nucleic acid sequence encoding the amino acid sequence of SEQ ID NO: 22 is set forth in SEQ ID NO: 23, which is provided below.

[SEQ ID NO: 23]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTTCAATGAACTGCAGAAAGATAAGAT
GGCGGAGGCCTTCAGTGAGATTGGGATGAAAGGCGAGCGCCGGAGGGGCA
AGGGGCACGATGGCCTTTTCCAGGGGCTCAGTACAGCCACCAAGGACACC
TTCGACGCCCTTCACATGCAGGCCCTGCCCCCTCGC

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of co-stimulatory molecules include CD28, 4-1BB, OX40, CD27, CD40, CD154, CD97, CD11a/CD18, ICOS, DAP-10, CD2, CD150, CD226, and NKG2D.

In certain embodiments, the intracellular signaling domain of the CAR comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the first antigen-recognizing receptor comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 7 or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 7, which is at least about 20, or at least about 30, or at least about 40, or at least about 50, and up to about 220 amino acids in length. Alternatively or additionally, in certain embodiments, the CD28 polypeptide comprised in the co-stimulatory signaling region of the CAR comprises or consists of amino acids 1 to 220, 1 to 50, 50 to 100, 100 to 150, 114 to 220, 150 to 200, 180 to 220, or 200 to 220 of SEQ ID NO: 7. In certain embodiments, the intracellular signaling domain of the CAR comprises a co-stimulatory signaling region that comprises a CD28 polypeptide comprising or consisting of amino acids 180 to 220 of SEQ ID NO: 7.

An exemplary nucleic acid sequence encoding the amino acid sequence of amino acids 180 to 220 of SEQ ID NO: 7 is set forth in SEQ ID NO: 24, which is provided below.

[SEQ ID NO: 24]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the intracellular signaling domain of the first antigen-recognizing receptor comprises a co-stimulatory signaling region that comprises an intracellular domain of mouse CD28 or a portion thereof. In certain embodiments, the CD28 polypeptide comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having a NCBI Reference No: NP_031668.3 (or SEQ ID NO: 25) or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 25, which is at least about 20, or at least about 30, or at least about 40, or at least about 50, and up to 218 amino acids in length. In certain embodiments, the CD28 polypeptide comprised in the co-stimulatory signaling region of the CAR comprises or consists of the amino acid sequence of amino acids 1 to 218, 1 to 50, 50 to 100, 100 to 150, 150 to 218, 178 to 218, or 200 to 218 of SEQ ID NO: 25. In certain embodiments, the co-stimulatory signaling region of the CAR comprises a CD28 polypeptide that comprises or consists of amino acids 178 to 218 of SEQ ID NO: 25. SEQ ID NO: 25 is provided below.

[SEQ ID NO: 25]
MTLRLLFLALNFFSVQVTENKILVKQSPLLVVDSNEVSLSCRYSYNLLAK
EFRASLYKGVNSDVEVCVGNGNFTYQPQFRSNAEFNCDGDFDNETVTERL
WNLHVNHTDIYFCKIEFMYPPPYLDNERSNGTIIHIKEKHLCHTQSSPKL
FWALVVVAGVLFCYGLLVTVALCVIWTNSRRNRLLQSDYMNMTPRRPGLT
RKPYQPYAPARDFAAYRP

In certain embodiments, the intracellular signaling domain of the CAR comprises a co-stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region of the CAR comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having a NCBI Ref. No.: NP_001552 (SEQ ID NO: 26) or a fragment thereof, and/or may optionally comprise up to one or up to two or up to three conservative amino acid substitutions. In certain embodiments, the 4-1BB comprised in the co-stimulatory signaling region of the CAR comprises or consists of an amino acid sequence that is a consecutive portion of SEQ ID NO: 26, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the co-stimulatory signaling region of the CAR comprises a 4-1BB polypeptide that comprises or consists of the amino acid sequence of amino acids 1 to 255, 1 to 50, 50 to 100, 100 to 150, 150 to 200, or 200 to 255 of SEQ ID NO: 26. In certain embodiments, the co-stimulatory signaling region of the CAR comprises a 4-1BB polypeptide comprising or consisting of the amino acid sequence of amino acids 214 to 255 of SEQ ID NO: 26. SEQ ID NO: 26 is provided below.

[SEQ ID NO: 26]
MGNSCYNIVATLLLVLNFERTRSLQDPCSNCPAGTFCDNNRNQICSPCPP
NSESSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

In certain embodiments, the intracellular signaling domain of the CAR comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, CD150, CD226, and NKG2D. In certain embodiments, the intracellular signaling domain of the CAR comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

In addition, the extracellular antigen-binding domain of the CAR can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the CAR. Exemplary leader sequences include, but is not limited to, a human IL-2 signal sequence (e.g., a human IL-2 signal sequence comprising or consisting of the amino acid sequence set forth in SEQ ID NO: 27), a mouse IL-2 signal sequence (e.g., a mouse IL-2 signal sequence comprising or consisting of the amino acid sequence set forth in SEQ ID NO: 28); a human kappa leader sequence (e.g., a human kappa leader sequence comprising or consisting of the amino acid sequence set forth in SEQ ID NO: 29), a mouse kappa leader sequence (e.g., a mouse kappa leader sequence comprising or consisting of the amino acid sequence set forth in SEQ ID NO: 30); a human CD8 leader sequence (e.g., a human CD8 leader sequence comprising or consisting of the amino acid sequence set forth in SEQ ID NO: 31); a truncated human CD8 signal peptide (e.g., a truncated human CD8 signal peptide comprising or consisting of the amino acid sequence set forth in SEQ ID NO: 32); a human albumin signal sequence (e.g., a human albumin signal sequence comprising or consisting of the amino acid sequence set forth in SEQ ID NO: 33); and a human prolactin signal sequence (e.g., a human prolactin signal sequence comprising or consisting of the amino acid sequence set forth in SEQ ID NO: 34). SEQ ID Nos: 27-34 are provided below.

[SEQ ID NO: 27]
MYRMQLLSCIALSLALVTNS
[SEQ ID NO: 28]
MYSMQLASCVTLTLVLLVNS
[SEQ ID NO: 29]
METPAQLLFLLLLWLPDTTG
[SEQ ID NO: 30]
METDTLLLWVLLLWVPGSTG
[SEQ ID NO: 31]
MALPVTALLLPLALLLHAARP
[SEQ ID NO: 32]
MALPVTALLLPLALLLHA
[SEQ ID NO: 33]
MKWVTFISLLFSSAYS
[SEQ ID NO: 34]
MDSKGSSQKGSRLLLLLVVSNLLLCQGVVS

In certain embodiments, the signal peptide comprises a CD8 polypeptide, e.g., the CAR comprises a truncated CD8 signal peptide. In certain embodiments, the signal peptide comprises or consists of the amino acid sequence set forth in SEQ ID NO: 31.

### 5.3.1.2.2. Chimeric Co-stimulating Receptors (CCRs)

In certain embodiments, a presently disclosed cell comprising a presently disclosed antigen-recognizing receptor further comprises chimeric co-stimulating receptor (CCR). The term “chimeric co-stimulating receptor” or “CCR” refers to a chimeric receptor that binds to an antigen and provides a co-stimulatory signal, but does not provide a T-cell activation signal to a cell comprising the CCR. Various CCRs are described in US20020018783 the contents of which are incorporated by reference in their entireties. CCRs mimic co-stimulatory signals, but unlike, CARs, do not provide a T-cell activation signal. In certain embodiments, the CCR lacks a CD35 polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013);31 (1): 71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to a second antigen and an intracellular domain that is capable of delivering a co-stimulatory signal to the cell but does not alone deliver an activation signal to the cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the CCR comprises an intracellular domain of CD28 or a portion thereof. In certain embodiments, the CCR comprises an intracellular domain of 4-1BB or a portion thereof. In certain embodiments, the CCR comprises an intracellular domain of CD28 or a portion thereof, and an intracellular domain of 4-1BB or a portion thereof.

In certain embodiments, the second antigen is selected so that expression of both a first antigen (e.g., antigen targeted by a CAR) and the second antigen is restricted to the targeted cells (e.g., cancerous tissue or cancerous cells, or LSCs, or AML HSPCs). Similar to a CAR, the extracellular antigen-binding domain can be an scFv, a Fab, a F(ab)₂, or a fusion protein with a heterologous sequence to form the extracellular antigen-binding domain.

In certain embodiments, the cell comprising a first antigen-recognizing receptor (e.g., a CAR) and a second antigen-recognizing receptor (e.g., a CAR, a TCR, or a TCR like fusion molecule) exhibits a greater degree of cytolytic activity against cells that are positive for both the first antigen and the second antigen as compared to against cells that are singly positive for the first antigen. In certain embodiments, the cell comprising the first antigen-recognizing receptor (e.g., a CAR) and the second antigen-recognizing receptor (e.g., a CAR, a TCR, or a TCR like fusion molecule) exhibits substantially no or negligible cytolytic activity against cells that are singly positive for the first antigen.

In certain embodiments, the first antigen recognizing receptor (e.g., a CAR, a TCR, or a TCR like fusion molecule) binds to the first antigen with a low binding affinity, e.g., a dissociation constant (K_{D}) of about 1×10⁻⁸ M or more, about 5×10⁻⁸ M or more, about 1×10⁻⁷ M or more, about 5×10⁻⁷ M or more, or about 1×10⁻⁶ M or more, or from about 1×10⁻⁸ M to about 1×10⁻⁶ M. In certain embodiments, the first antigen recognizing receptor (e.g., a CAR, a TCR, or a TCR like fusion molecule) binds to the first antigen with a low binding avidity. In certain embodiments, the first antigen recognizing receptor (e.g., a CAR, a TCR, or a TCR like fusion molecule) binds to the first antigen at an epitope of low accessibility. In certain embodiments, the first antigen recognizing receptor (e.g., a CAR, a TCR, or a TCR like fusion molecule) binds to the first antigen with a binding affinity that is lower compared to the binding affinity with which the second antigen-recognizing receptor (e.g., a CCR) binds to the second antigen. In certain embodiments, the CCR binds to the second antigen with a binding affinity K_{D} of from about 1×10⁻⁹ M to about 1×10⁻⁷ M, e.g., about 1×10⁻⁷ M or less, about 1×10⁻⁸ M or less, or about 1×10⁻⁹ M or less.

### 5.3.1.3. T Cell Receptors (TCRs)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the antigen-recognizing receptor is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 5.3.1.4. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRUC receptors engaging the complete T cell receptor for potent anti-tumor response,” Nature Communications volume 10, Article number: 2087 (2019), which is incorporated by reference in its entirety).

In certain embodiments, the TCR like fusion molecule is a recombinant T cell receptor (TCR). In certain embodiments, the recombinant TCR comprises at least one antigen-binding chain. In certain embodiments, the antigen-binding domain of the recombinant TCR comprises a ligand for a cell-surface receptor, a receptor for a cell surface ligand, an antigen binding portion of an antibody or a fragment thereof, or an antigen binding portion of a TCR. In certain embodiments, the recombinant TCR comprises two antigen binding chains, i.e., a first antigen binding chain and a second antigen binding chain. In certain embodiments, the first and second antigen-binding chains each comprises a constant domain. In certain embodiments, the recombinant TCR binds to an antigen (e.g., a first antigen or a second antigen) in an HLA-independent manner. Thus, in certain embodiments, the recombinant TCR is an HLA-independent (or non-HLA restricted) TCR (referred to as “HIT-CAR” or “HIT”).

In certain embodiments, the first antigen-binding chain comprises an antigen-binding fragment of a heavy chain variable region (V_{H}) of an antibody. In certain embodiments, the second antigen-binding chain comprises an antigen-binding fragment of a light chain variable region (V_{L}) of an antibody. In certain embodiments, the first antigen-binding chain comprises an antigen-binding fragment of a V_{H} of an antibody, and the second antigen-binding chain comprises an antigen-binding fragment of a V_{L} of the antibody.

In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the first antigen-binding chain comprises a TRAC polypeptide, and the second antigen-binding chain comprises a TRBC polypeptide. In certain embodiments, the first antigen-binding chain comprises a TRBC polypeptide, and the second antigen-binding chain comprises a TRAC polypeptide.

In certain embodiments, the first antigen-binding chain comprises a V_{H} of an antibody and a TRAC polypeptide, and the second antigen-binding chain comprises a V_{L} of an antibody and a TRBC polypeptide.

In certain embodiments, the first antigen-binding chain comprises a V_{H} of an antibody and a TRBC polypeptide, and the second antigen-binding chain comprises a V_{L} of an antibody and a TRAC polypeptide.

In certain embodiments, at least one of the TRAC polypeptide and the TRBC polypeptide is endogenous. In certain embodiments, the TRAC polypeptide is endogenous. In certain embodiments, the TRBC polypeptide is endogenous. In certain embodiments, both the TRAC polypeptide and the TRBC polypeptide are endogenous.

In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the TRAC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by the gene of NCBI Genbank ID: 28755, NG_001332.3, range 925603 to 930229 (SEQ ID NO: 35) or a fragment thereof, and/or may optionally comprise up to one or up to two or up to three conservative amino acid substitutions. In certain embodiments, the TRAC polypeptide comprises or consists of the amino acid sequence encoded by the nucleotide sequence of SEQ ID NO: 35. SEQ ID NO: 35 is provided below.

[SEQ ID NO: 35]
ATATCCAGAACCCTGACCCTGCCGTGTACCAGCTGAGAGACTCTAAATCCAGTGACAAGT
CTGTCTGCCTATTCACCGATTTTGATTCTCAAACAAATGTGTCACAAAGTAAGGATTCTG
ATGTGTATATCACAGACAAAACTGTGCTAGACATGAGGTCTATGGACTTCAAGAGCAACA
GTGCTGTGGCCTGGAGCAACAAATCTGACTTTGCATGTGCAAACGCCTTCAACAACAGCA
TTATTCCAGAAGACACCTTCTTCCCCAGCCCAGGTAAGGGCAGCTTTGGTGCCTTCGCAG
GCTGTTTCCTTGCTTCAGGAATGGCCAGGTTCTGCCCAGAGCTCTGGTCAATGATGTCTA
AAACTCCTCTGATTGGTGGTCTCGGCCTTATCCATTGCCACCAAAACCCTCTTTTTACTA
AGAAACAGTGAGCCTTGTTCTGGCAGTCCAGAGAATGACACGGGAAAAAAGCAGATGAAG
AGAAGGTGGCAGGAGAGGGCACGTGGCCCAGCCTCAGTCTCTCCAACTGAGTTCCTGCCT
GCCTGCCTTTGCTCAGACTGTTTGCCCCTTACTGCTCTTCTAGGCCTCATTCTAAGCCCC
TTCTCCAAGTTGCCTCTCCTTATTTCTCCCTGTCTGCCAAAAAATCTTTCCCAGCTCACT
AAGTCAGTCTCACGCAGTCACTCATTAACCCACCAATCACTGATTGTGCCGGCACATGAA
TGCACCAGGTGTTGAAGTGGAGGAATTAAAAAGTCAGATGAGGGGTGTGCCCAGAGGAAG
CACCATTCTAGTTGGGGGAGCCCATCTGTCAGCTGGGAAAAGTCCAAATAACTTCAGATT
GGAATGTGTTTTAACTCAGGGTTGAGAAAACAGCTACCTTCAGGACAAAAGTCAGGGAAG
GGCTCTCTGAAGAAATGCTACTTGAAGATACCAGCCCTACCAAGGGCAGGGAGAGGACCC
TATAGAGGCCTGGGACAGGAGCTCAATGAGAAAGGAGAAGAGCAGCAGGCATGAGTTGAA
TGAAGGAGGCAGGGCCGGGTCACAGGGCCTTCTAGGCCATGAGAGGGTAGACAGTATTCT
AAGGACGCCAGAAAGCTGTTGATCGGCTTCAAGCAGGGGAGGGACACCTAATTTGCTTTT
CTTTTTTTTTTTTTTTTTTTTTTTTTTTTTTGAGATGGAGTTTTGCTCTTGTTGCCCAGG
CTGGAGTGCAATGGTGCATCTTGGCTCACTGCAACCTCCGCCTCCCAGGTTCAAGTGATT
CTCCTGCCTCAGCCTCCCGAGTAGCTGAGATTACAGGCACCCGCCACCATGCCTGGCTAA
TTTTTTGTATTTTTAGTAGAGACAGGGTTTCACTATGTTGGCCAGGCTGGTCTCGAACTC
CTGACCTCAGGTGATCCACCCGCTTCAGCCTCCCAAAGTGCTGGGATTACAGGCGTGAGC
CACCACACCCGGCCTGCTTTTCTTAAAGATCAATCTGAGTGCTGTACGGAGAGTGGGTTG
TAAGCCAAGAGTAGAAGCAGAAAGGGAGCAGTTGCAGCAGAGAGATGATGGAGGCCTGGG
CAGGGTGGTGGCAGGGAGGTAACCAACACCATTCAGGTTTCAAAGGTAGAACCATGCAGG
GATGAGAAAGCAAAGAGGGGATCAAGGAAGGCAGCTGGATTTTGGCCTGAGCAGCTGAGT
CAATGATAGTGCCGTTTACTAAGAAGAAACCAAGGAAAAAATTTGGGGTGCAGGGATCAA
AACTTTTTGGAACATATGAAAGTACGTGTTTATACTCTTTATGGCCCTTGTCACTATGTA
TGCCTCGCTGCCTCCATTGGACTCTAGAATGAAGCCAGGCAAGAGCAGGGTCTATGTGTG
ATGGCACATGTGGCCAGGGTCATGCAACATGTACTTTGTACAAACAGTGTATATTGAGTA
AATAGAAATGGTGTCCAGGAGCCGAGGTATCGGTCCTGCCAGGGCCAGGGGCTCTCCCTA
GCAGGTGCTCATATGCTGTAAGTTCCCTCCAGATCTCTCCACAAGGAGGCATGGAAAGGC
TGTAGTTGTTCACCTGCCCAAGAACTAGGAGGTCTGGGGTGGGAGAGTCAGCCTGCTCTG
GATGCTGAAAGAATGTCTGTTTTTCCTTTTAGAAAGTTCCTGTGATGTCAAGCTGGTCGA
GAAAAGCTTTGAAACAGGTAAGACAGGGGTCTAGCCTGGGTTTGCACAGGATTGCGGAAG
TGATGAACCCGCAATAACCCTGCCTGGATGAGGGAGTGGGAAGAAATTAGTAGATGTGGG
AATGAATGATGAGGAATGGAAACAGCGGTTCAAGACCTGCCCAGAGCTGGGTGGGGTCTC
TCCTGAATCCCTCTCACCATCTCTGACTTTCCATTCTAAGCACTTTGAGGATGAGTTTCT
AGCTTCAATAGACCAAGGACTCTCTCCTAGGCCTCTGTATTCCTTTCAACAGCTCCACTG
TCAAGAGAGCCAGAGAGAGCTTCTGGGTGGCCCAGCTGTGAAATTTCTGAGTCCCTTAGG
GATAGCCCTAAACGAACCAGATCATCCTGAGGACAGCCAAGAGGTTTTGCCTTCTTTCAA
GACAAGCAACAGTACTCACATAGGCTGTGGGCAATGGTCCTGTCTCTCAAGAATCCCCTG
CCACTCCTCACACCCACCCTGGGCCCATATTCATTTCCATTTGAGTTGTTCTTATTGAGT
CATCCTTCCTGTGGTAGCGGAACTCACTAAGGGGCCCATCTGGACCCGAGGTATTGTGAT
GATAAATTCTGAGCACCTACCCCATCCCCAGAAGGGCTCAGAAATAAAATAAGAGCCAAG
TCTAGTCGGTGTTTCCTGTCTTGAAACACAATACTGTTGGCCCTGGAAGAATGCACAGAA
TCTGTTTGTAAGGGGATATGCACAGAAGCTGCAAGGGACAGGAGGTGCAGGAGCTGCAGG
CCTCCCCCACCCAGCCTGCTCTGCCTTGGGGAAAACCGTGGGTGTGTCCTGCAGGCCATG
CAGGCCTGGGACATGCAAGCCCATAACCGCTGTGGCCTCTTGGTTTTACAGATACGAACC
TAAACTTTCAAAACCTGTCAGTGATTGGGTTCCGAATCCTCCTCCTGAAAGTGGCCGGGT
TTAATCTGCTCATGACGCTGCGGCTGTGGTCCAGCTGAGGTGAGGGGCCTTGAAGCTGGG
AGTGGGGTTTAGGGACGCGGGTCTCTGGGTGCATCCTAAGCTCTGAGAGCAAACCTCCCT
GCAGGGTCTTGCTTTTAAGTCCAAAGCCTGAGCCCACCAAACTCTCCTACTTCTTCCTGT
TACAAATTCCTCTTGTGCAATAATAATGGCCTGAAACGCTGTAAAATATCCTCATTTCAG
CCGCCTCAGTTGCACTTCTCCCCTATGAGGTAGGAAGAACAGTTGTTTAGAAACGAAGAA
ACTGAGGCCCCACAGCTAATGAGTGGAGGAAGAGAGACACTTGTGTACACCACATGCCTT
GTGTTGTACTTCTCTCACCGTGTAACCTCCTCATGTCCTCTCTCCCCAGTACGGCTCTCT
TAGCTCAGTAGAAAGAAGACATTACACTCATATTACACCCCAATCCTGGCTAGAGTCTCC
GCACCCTCCTCCCCCAGGGTCCCCAGTCGTCTTGCTGACAACTGCATCCTGTTCCATCAC
CATCAAAAAAAAACTCCAGGCTGGGTGCGGGGGCTCACACCTGTAATCCCAGCACTTTGG
GAGGCAGAGGCAGGAGGAGCACAGGAGCTGGAGACCAGCCTGGGCAACACAGGGAGACCC
CGCCTCTACAAAAAGTGAAAAAATTAACCAGGTGTGGTGCTGCACACCTGTAGTCCCAGC
TACTTAAGAGGCTGAGATGGGAGGATCGCTTGAGCCCTGGAATGTTGAGGCTACAATGAG
CTGTGATTGCGTCACTGCACTCCAGCCTGGAAGACAAAGCAAGATCCTGTCTCAAATAAT
AAAAAAAATAAGAACTCCAGGGTACATTTGCTCCTAGAACTCTACCACATAGCCCCAAAC
AGAGCCATCACCATCACATCCCTAACAGTCCTGGGTCTTCCTCAGTGTCCAGCCTGACTT
CTGTTCTTCCTCATTCCAGATCTGCAAGATTGTAAGACAGCCTGTGCTCCCTCGCTCCTT
CCTCTGCATTGCCCCTCTTCTCCCTCTCCAAACAGAGGGAACTCTCCTACCCCCAAGGAG
GTGAAAGCTGCTACCACCTCTGTGCCCCCCCGGCAATGCCACCAACTGGATCCTACCCGA
ATTTATGATTAAGATTGCTGAAGAGCTGCCAAACACTGCTGCCACCCCCTCTGTTCCCTT
ATTGCTGCTTGTCACTGCCTGACATTCACGGCAGAGGCAAGGCTGCTGCAGCCTCCCCTG
GCTGTGCACATTCCCTCCTGCTCCCCAGAGACTGCCTCCGCCATCCCACAGATGATGGAT
CTTCAGTGGGTTCTCTTGGGCTCTAGGTCCTGCAGAATGTTGTGAGGGGTTTATTTTTTT
TTAATAGTGTTCATAAAGAAATACATAGTATTCTTCTTCTCAAGACGTGGGGGGAAATTA
TCTCATTATCGAGGCCCTGCTATGCTGTGTATCTGGGCGTGTTGTATGTCCTGCTGCCGA
TGCCTTC

In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 36 or a fragment thereof, and/or may optionally comprise up to one or up to two or up to three conservative amino acid substitutions.

In certain embodiments, the TRBC2 polypeptide comprises or consists of the amino acid sequence set forth in SEQ ID NO: 36. SEQ ID NO: 36 is provided below.

[SEQ ID NO: 36]
DLKNVFPPEVAVFEPSEAEISHTQKATLVCLATGFYPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSESYQQGVLSATILYEI
LLGKATLYAVLVSALVLMAMVKRKDSRG

An exemplary nucleotide sequence encoding the amino acid sequence of SEQ ID NO: 36 is set forth in SEQ ID NO: 37, which is provided below.

[SEQ ID NO: 37]
GATCTGAAAAACGTGTTCCCTCCTGAAGTGGCTGTCTTTGAACCATCCGA
GGCCGAGATTTCCCATACCCAGAAAGCAACTCTGGTCTGTCTGGCCACTG
GATTCTACCCCGATCACGTGGAACTGTCTTGGTGGGTGAACGGCAAGGAA
GTCCATTCCGGAGTCTCTACCGACCCTCAGCCCCTCAAGGAGCAGCCTGC
TCTCAACGATTCTCGGTACTGCCTGTCATCTCGACTGAGAGTGTCTGCCA
CCTTCTGGCAGAACCCTAGAAACCACTTTCGGTGTCAGGTCCAGTTTTAC
GGCCTGAGCGAGAACGATGAGTGGACACAGGATAGAGCCAAACCTGTGAC
ACAGATTGTGAGCGCCGAGGCTTGGGGACGAGCCGATTGTGGCTTCACAT
CCGAGTCTTACCAGCAGGGAGTGCTGTCTGCTACAATCCTCTACGAAATT
CTCCTGGGGAAGGCCACCCTGTACGCTGTCCTCGTGTCTGCTCTGGTGCT
CATGGCTATGGTCAAACGAAAGGACTCTAGAGGC

In certain embodiments, the TRBC polypeptide is a TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 38 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 38. SEQ ID NO: 38 is provided below.

[SEQ ID NO: 38]
LNKVFPPEVAVFEPSEAEISHTQKATLVCLATGFFPDHVELSWWVNGKE
VHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILY
EILLGKATLYAVLVSALVLMAMVKRKDF

In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 39 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 39. SEQ ID NO: 39 is provided below.

[SEQ ID NO: 39]
DLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQ
FYGLSENDEWTQDRAKPVTQIVSAEAWGRADCGFTSVSYQQGVLSATIL
YEILLGKATLYAVLVSALVLMAMVKRKDF

An exemplary nucleotide sequence encoding the amino acid sequence of SEQ ID NO: 39 is set forth in SEQ ID NO: 40, which is provided below.

[SEQ ID NO: 40]
GACCTGAACAAGGTGTTCCCACCCGAGGTCGCTGTGTTTGAGCCATCAG
AAGCAGAGATCTCCCACACCCAAAAGGCCACACTGGTGTGCCTGGCCAC
AGGCTTCTTCCCCGACCACGTGGAGCTGAGCTGGTGGGTGAATGGGAAG
GAGGTGCACAGTGGGGTCAGCACAGACCCGCAGCCCCTCAAGGAGCAGC
CCGCCCTCAATGACTCCAGATACTGCCTGAGCAGCCGCCTGAGGGTCTC
GGCCACCTTCTGGCAGAACCCCCGCAACCACTTCCGCTGTCAAGTCCAG
TTCTACGGGCTCTCGGAGAATGACGAGTGGACCCAGGATAGGGCCAAAC
CCGTCACCCAGATCGTCAGCGCCGAGGCCTGGGGTAGAGCAGACTGTGG
CTTTACCTCGGTGTCCTACCAGCAAGGGGTCCTGTCTGCCACCATCCTC
TATGAGATCCTGCTAGGGAAGGCCACCCTGTATGCTGTGCTGGTCAGCG
CCCTTGTGTTGATGGCCATGGTCAAGAGAAAGGATTTC

In certain embodiments, the TRBC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by a gene of NCBI GenBank ID: 28639, NG_001333.2, range 645749 to 647196 (TRBC1, SEQ ID NO: 41), NCBI GenBank ID: 28638, NG_001333.2 range 655095 to 656583 (TRBC2, SEQ ID NO:42) or a fragment thereof, and/or may optionally comprise up to one or up to two or up to three conservative amino acid substitutions. In certain embodiments, the TRBC polypeptide comprises or consists of the amino acid sequence encoded by the nucleotide sequence of SEQ ID NO: 41. In certain embodiments, the TRBC polypeptide comprises or consists of the amino acid sequence encoded by the nucleotide sequence of SEQ ID NO: 42. SEQ ID NOs: 41 and 42 are provided below.

[SEQ ID NO: 41]
AGGACCTGAACAAGGTGTTCCCACCCGAGGTCGCTGTGTTTGAGCCATC
AGAAGCAGAGATCTCCCACACCCAAAAGGCCACACTGGTGTGCCTGGCC
ACAGGCTTCTTCCCCGACCACGTGGAGCTGAGCTGGTGGGTGAATGGGA
AGGAGGTGCACAGTGGGGTCAGCACAGACCCGCAGCCCCTCAAGGAGCA
GCCCGCCCTCAATGACTCCAGATACTGCCTGAGCAGCCGCCTGAGGGTC
TCGGCCACCTTCTGGCAGAACCCCCGCAACCACTTCCGCTGTCAAGTCC
AGTTCTACGGGCTCTCGGAGAATGACGAGTGGACCCAGGATAGGGCCAA
ACCCGTCACCCAGATCGTCAGCGCCGAGGCCTGGGGTAGAGCAGGTGAG
TGGGGCCTGGGGAGATGCCTGGAGGAGATTAGGTGAGACCAGCTACCAG
GGAAAATGGAAAGATCCAGGTAGCAGACAAGACTAGATCCAAAAAGAAA
GGAACCAGCGCACACCATGAAGGAGAATTGGGCACCTGTGGTTCATTCT
TCTCCCAGATTCTCAGCCCAACAGAGCCAAGCAGCTGGGTCCCCTTTCT
ATGTGGCCTGTGTAACTCTCATCTGGGTGGTGCCCCCCATCCCCCTCAG
TGCTGCCACATGCCATGGATTGCAAGGACAATGTGGCTGACATCTGCAT
GGCAGAAGAAAGGAGGTGCTGGGCTGTCAGAGGAAGCTGGTCTGGGCCT
GGGAGTCTGTGCCAACTGCAAATCTGACTTTACTTTTAATTGCCTATGA
AAATAAGGTCTCTCATTTATTTTCCTCTCCCTGCTTTCTTTCAGACTGT
GGCTTTACCTCGGGTAAGTAAGCCCTTCCTTTTCCTCTCCCTCTCTCAT
GGTTCTTGACCTAGAACCAAGGCATGAAGAACTCACAGACACTGGAGGG
TGGAGGGTGGGAGAGACCAGAGCTACCTGTGCACAGGTACCCACCTGTC
CTTCCTCCGTGCCAACAGTGTCCTACCAGCAAGGGGTCCTGTCTGCCAC
CATCCTCTATGAGATCCTGCTAGGGAAGGCCACCCTGTATGCTGTGCTG
GTCAGCGCCCTTGTGTTGATGGCCATGGTAAGCAGGAGGGCAGGATGGG
GCCAGCAGGCTGGAGGTGACACACTGACACCAAGCACCCAGAAGTATAG
AGTCCCTGCCAGGATTGGAGCTGGGCAGTAGGGAGGGAAGAGATTTCAT
TCAGGTGCCTCAGAAGATAACTTGCACCTCTGTAGGATCACAGTGGAAG
GGTCATGCTGGGAAGGAGAAGCTGGAGTCACCAGAAAACCCAATGGATG
TTGTGATGAGCCTTACTATTTGTGTGGTCAATGGGCCCTACTACTTTCT
CTCAATCCTCACAACTCCTGGCTCTTAATAACCCCCAAAACTTTCTCTT
CTGCAGGTCAAGAGAAAGGATTTCTGA
[SEQ ID NO: 42]
AGGACCTGAAAAACGTGTTCCCACCCGAGGTCGCTGTGTTTGAGCCATC
AGAAGCAGAGATCTCCCACACCCAAAAGGCCACACTGGTATGCCTGGCC
ACAGGCTTCTACCCCGACCACGTGGAGCTGAGCTGGTGGGTGAATGGGA
AGGAGGTGCACAGTGGGGTCAGCACAGACCCGCAGCCCCTCAAGGAGCA
GCCCGCCCTCAATGACTCCAGATACTGCCTGAGCAGCCGCCTGAGGGTC
TCGGCCACCTTCTGGCAGAACCCCCGCAACCACTTCCGCTGTCAAGTCC
AGTTCTACGGGCTCTCGGAGAATGACGAGTGGACCCAGGATAGGGCCAA
ACCCGTCACCCAGATCGTCAGCGCCGAGGCCTGGGGTAGAGCAGGTGAG
TGGGGCCTGGGGAGATGCCTGGAGGAGATTAGGTGAGACCAGCTACCAG
GGAAAATGGAAAGATCCAGGTAGCGGACAAGACTAGATCCAGAAGAAAG
CCAGAGTGGACAAGGTGGGATGATCAAGGTTCACAGGGTCAGCAAAGCA
CGGTGTGCACTTCCCCCACCAAGAAGCATAGAGGCTGAATGGAGCACCT
CAAGCTCATTCTTCCTTCAGATCCTGACACCTTAGAGCTAAGCTTTCAA
GTCTCCCTGAGGACCAGCCATACAGCTCAGCATCTGAGTGGTGTGCATC
CCATTCTCTTCTGGGGTCCTGGTTTCCTAAGATCATAGTGACCACTTCG
CTGGCACTGGAGCAGCATGAGGGAGACAGAACCAGGGCTATCAAAGGAG
GCTGACTTTGTACTATCTGATATGCATGTGTTTGTGGCCTGTGAGTCTG
TGATGTAAGGCTCAATGTCCTTACAAAGCAGCATTCTCTCATCCATTTT
TCTTCCCCTGTTTTCTTTCAGACTGTGGCTTCACCTCCGGTAAGTGAGT
CTCTCCTTTTTCTCTCTATCTTTCGCCGTCTCTGCTCTCGAACCAGGGC
ATGGAGAATCCACGGACACAGGGGCGTGAGGGAGGCCAGAGCCACCTGT
GCACAGGTGCCTACATGCTCTGTTCTTGTCAACAGAGTCTTACCAGCAA
GGGGTCCTGTCTGCCACCATCCTCTATGAGATCTTGCTAGGGAAGGCCA
CCTTGTATGCCGTGCTGGTCAGTGCCCTCGTGCTGATGGCCATGGTAAG
GAGGAGGGTGGGATAGGGCAGATGATGGGGGCAGGGGATGGAACATCAC
ACATGGGCATAAAGGAATCTCAGAGCCAGAGCACAGCCTAATATATCCT
ATCACCTCAATGAAACCATAATGAAGCCAGACTGGGGAGAAAATGCAGG
AGAATATCACAGAATGCATCATGGGAGGATGGAGACAACCAGCGAGCCC
TACTCAAATTAGGCCTCAGAGCCCGCCTCCCCTGCCCTACTCCTGCTGT
GCCATAGCCCCTGAAACCCTGAAAATGTTCTCTCTTCCACAGGTCAAGA
GAAAGGATTCCAGAGGCTAG

In certain embodiments, the TCR like fusion molecule comprises a hinge/spacer region that links the first antigen binding chain to the constant domain. In certain embodiments, the TCR like fusion molecule comprises a hinge/spacer region that links the second antigen binding chain to the constant domain. The hinge/spacer region can be flexible enough to allow the antigen binding chain to orient in different directions to facilitate antigen recognition. In certain embodiments, the hinge/spacer region can be the hinge region from IgG1, the C_{H}2C_{H}3 region of immunoglobulin and portions of CD3, a portion of a TCRα polypeptide, a portion of a TCRβ polypeptide, a portion of a CD28 polypeptide, a portion of a CD8 polypeptide, or a synthetic spacer sequence. In certain embodiments, the hinge/spacer region comprises a portion of a TCRα polypeptide. In certain embodiments, the hinge/spacer region comprises a portion of the variable region (TRAV), a portion of the diversity region (TRAD), a portion of the joining region (TRAJ), a portion of the constant region (TRAC), or a combination thereof. In certain embodiments, the hinge/spacer region comprises a portion of the TRAJ region and a portion of the TRAC region of the TCRα polypeptide. In certain embodiments, the hinge/spacer region comprises or consists of the amino acid sequence set forth in SEQ ID NO: 43. In certain embodiments, the hinge/spacer region comprises or consists of amino acids 1 to 3 of the sequence set forth in SEQ ID NO: 43. An exemplary nucleic acid sequence encoding the amino acid sequence of SEQ ID NO: 43 is set forth in SEQ ID NO: 44. SEQ ID Nos: 43 and 44 are provided below.

[SEQ ID NO: 43]
IPNIQNPDPA
[SEQ ID NO: 44]
ATTCCCAATATCCAGAACCCTGACCCTGCC

In certain embodiments, the hinge/spacer region comprises a portion of a TCRβ polypeptide. In certain embodiments, the hinge/spacer region comprises a portion of the variable region (TRBV), a portion of the diversity region (TRBD), a portion of the joining region (TRBJ), a portion of the constant region (TRBC), or a combination thereof. In certain embodiments, the hinge/spacer region comprises a portion of the TRBJ region and a portion of the TRAC region (C) of the TCRβ polypeptide. In certain embodiments, the hinge/spacer region comprises or consists of the amino acid sequence set forth in SEQ ID NO: 57. In certain embodiments, the hinge/spacer region comprises or consists of amino acid 1 to 2 of the sequence set forth in SEQ ID NO: 57. An exemplary nucleic acid sequence encoding the amino acid sequence of SEQ ID NO: 57 is set forth in SEQ ID NO: 58. SEQ ID Nos: 57 and 58 are provided below.

[SEQ ID NO: 57]
LEDLKNVFPPE
[SEQ ID NO: 58]
CTGGAGGATCTGAAAAACGTGTTCCCTCCTGAA

In certain embodiments, the antigen binding chain does not comprise an intracellular domain. In certain embodiments, the antigen binding chain is capable of associating with a CD3ζ polypeptide. In certain embodiments, the antigen binding chain associating with the CD3ζ polypeptide via the constant domain. In certain embodiments, the CD3ζ polypeptide is endogenous. In certain embodiments, the CD3ζ polypeptide is exogenous. In certain embodiments, binding of the antigen binding chain to a target antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the CD3ζ polypeptide comprises or consists of an amino acid sequence that is at least about 85%, about 90%, about 95%, about 96%, about 97%, about 98%, about 99% or about 100% homologous to the amino acid sequence set forth in SEQ ID NO: 8 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 8, which is at least about 20, or at least about 30, or at least about 40, or at least about 50, and up to about 164 amino acids in length. In certain embodiments, the CD3ζ comprises or consists of the amino acid sequence of amino acids 1 to 164, 1 to 50, 50 to 100, 52 to 164, 100 to 150, or 150 to 164 of SEQ ID NO: 8. In certain embodiments, the CD3ζ polypeptide comprises or consists of amino acids 52 to 164 of SEQ ID NO: 8.

In certain embodiments, the CD3ζ polypeptide comprises or consists of an amino acid sequence that is at least about 85%, about 90%, about 95%, about 96%, about 97%, about 98%, about 99% or about 100% homologous or identical to SEQ ID NO: 12 or a fragment thereof, and/or may optionally comprise up to one or up to two or up to three conservative amino acid substitutions. In certain embodiments, the CD3ζ polypeptide comprises or consists of the amino acid sequence set forth in SEQ ID NO: 12.

In certain embodiments, the TCR like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3ζ polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR like fusion molecule is capable of associating with a CD3 complex (also known as “T-cell co-receptor”). In certain embodiments, the TCR like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR like fusion molecule replaces a native and/or an endogenous TCR in the CD3/TCR complex. In certain embodiments, the CD3 complex comprises a CD3γ chain, a CD3ζ chain, and two CD3& chains.

In certain embodiments, the CD3γ chain comprises or consists of an amino acid sequence that is at least about 85%, about 90%, about 95%, about 96%, about 97%, about 98%, about 99% or about 100% homologous or identical to the amino acid sequence having a NCBI reference number: NP_000064.1 (SEQ ID NO: 45) or a fragment thereof, and/or may optionally comprise up to one or up to two or up to three conservative amino acid substitutions. SEQ ID NO: 45 is provided below.

[SEQ ID NO: 45]
MEQGKGLAVLILAIILLQGTLAQSIKGNHLVKVYDYQEDGSVLLTCDAE
AKNITWFKDGKMIGFLTEDKKKWNLGSNAKDPRGMYQCKGSQNKSKPLQ
VYYRMCQNCIELNAATISGFLFAEIVSIFVLAVGVYFIAGQDGVRQSRA
SDKQTLLPNDQLYQPLKDREDDQYSHLQGNQLRRN

In certain embodiments, the CD3δ chain comprises or consists of an amino acid sequence that is at least about 85%, about 90%, about 95%, about 96%, about 97%, about 98%, about 99% or about 100% homologous or identical to the amino acid sequence having a NCBI reference numbers: NP_000723.1 (SEQ ID NO: 46) or a fragment thereof, or the amino acid sequence having a NCBI reference numbers: NP_001035741.1 (SEQ ID NO: 47) or a fragment thereof, and/or may optionally comprise up to one or up to two or up to three conservative amino acid substitutions. SEQ ID NOS: 46 and 47 are provided below.

[SEQ ID NO: 46]
MEHSTFLSGLVLATLLSQVSPFKIPIEELEDRVFVNCNTSITWVEGTVG
TLLSDITRLDLGKRILDPRGIYRCNGTDIYKDKESTVQVHYRMCQSCVE
LDPATVAGIIVTDVIATLLLALGVFCFAGHETGRLSGAADTQALLRNDQ
VYQPLRDRDDAQYSHLGGNWARNK
[SEQ ID NO: 47]
MEHSTFLSGLVLATLLSQVSPFKIPIEELEDRVFVNCNTSITWVEGTVG
TLLSDITRLDLGKRILDPRGIYRCNGTDIYKDKESTVQVHYRTADTQAL
LRNDQVYQPLRDRDDAQYSHLGGNWARNK

In certain embodiments, the CD3ε chain comprises or consists of an amino acid sequence that is at least about 85%, about 90%, about 95%, about 96%, about 97%, about 98%, about 99% or about 100% homologous or identical to the amino acid sequence having a NCBI reference number: NP_000724.1 (SEQ ID NO: 48) or a fragment thereof, and/or may optionally comprise up to one or up to two or up to three conservative amino acid substitutions. SEQ ID NO: 48 is provided below.

[SEQ ID NO: 48]
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI

In certain embodiments, the TCR like fusion molecule exhibits a greater antigen sensitivity than a CAR targeting the same antigen. In certain embodiments, the TCR like fusion molecule is capable of inducing an immune response when binding to an antigen that has a low density on the surface of a tumor cell. In certain embodiments, cells comprising the TCR like fusion molecule can be used to treat a subject having tumor cells with a low expression level of a surface antigen, e.g., from a relapse of a disease, wherein the subject received treatment that leads to residual tumor cells. In certain embodiments, the tumor cells have a low density of a target molecule on the surface of the tumor cells. In certain embodiments, a target molecule having a low density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low density on the cell surface has a density of less than about 2,000 molecules per cell. In certain embodiments, a target molecule having a low density on the cell surface has a density of less than about 1,500 molecules per cell. In certain embodiments, a target molecule having a low density on the cell surface has a density of less than about 1,000 molecules per cell. In certain embodiments, a target molecule having a low density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

Various TCR like fusion molecules are disclosed in International Patent Application Publication No. WO2019/133969, which is incorporated by reference hereby in its entirety.

### 5.3.2. Co-stimulatory Ligands

In certain embodiments, a presently disclosed cell comprising a presently disclosed antigen-recognizing receptor further comprises at least one recombinant or exogenous co-stimulatory ligand. For example, a presently disclosed cell can be further transduced with at least one co-stimulatory ligand, such that the cell expresses or is induced to express the first antigen-recognizing receptor and the at least one co-stimulatory ligand. The at least one co-stimulatory ligand provides a co-stimulation signal to the cell.

Non-limiting examples of co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily shareseveral common features. The majority of TNF superfamily members are synthesized as type II transmembrane proteins (extracellular C-terminus) containing a short cytoplasmic segment and a relatively long extracellular region. Non-limiting examples of TNF superfamily members include nerve growth factor (NGF), CD40L (also known as “CD154”), 4-1BBL, TNF-α, OX40L, CD70, Fas ligand (FasL), CD30L, tumor necrosis factor beta (TNFβ)/lymphotoxin-alpha (LTa), lymphotoxin-beta (LTB), CD257/B cell-activating factor (BAFF)/Blys/THANK/Tall-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins-they possess an immunoglobulin domain (fold). Non-limiting examples of immunoglobulin superfamily ligands include CD80, CD86, and ICOSLG. In certain embodiments, the at least one co-stimulatory ligand is selected from the group consisting of 4-1BBL, CD80, CD86, CD70, GITRL, CD40L, OX40L, CD30L, TNFRSF14, ICOSLG, TRAIL, and combinations thereof.

In certain embodiments, the cell further comprises one exogenous co-stimulatory ligand that is 4-1BBL. In certain embodiments, the co-stimulatory ligand is human 4-1BBL. In certain embodiments, the 4-1BBL comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a Uniprot Reference No: P41273-1 (SEQ ID NO: 49) or a fragment thereof, and/or may optionally comprise up to one or up to two or up to three conservative amino acid substitutions. In certain embodiments, the 4-1BBL comprises or consists of an amino acid sequence that is a consecutive portion of the amino acid sequence of SEQ ID NO: 49. SEQ ID NO: 49 is provided below.

[SEQ ID NO: 49]
MEYASDASLDPEAPWPPAPRARACRVLPWALVAGLLLLLLLAAACAVFL
ACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSA
FGFQGRLLHLSAGQRLGVHLHTEARARHAWQLTQGATVLGLFRVTPEIP
AGLPSPRSE

An exemplary nucleotide sequence encoding the amino acid sequence of SEQ ID NO: 49 is set forth in SEQ ID NO: 50.

[SEQ ID NO: 50]
ATGGAATACGCCTCTGACGCTTCACTGGACCCCGAAGCCCCGTGGCCTC
CCGCGCCCCGCGCTCGCGCCTGCCGCGTACTGCCTTGGGCCCTGGTCGC
GGGGCTGCTGCTGCTGCTGCTGCTCGCTGCCGCCTGCGCCGTCTTCCTC
GCCTGCCCCTGGGCCGTGTCCGGGGCTCGCGCCTCGCCCGGCTCCGCGG
CCAGCCCGAGACTCCGCGAGGGTCCCGAGCTTTCGCCCGACGATCCCGC
CGGCCTCTTGGACCTGCGGCAGGGCATGTTTGCGCAGCTGGTGGCCCAA
AATGTTCTGCTGATCGATGGGCCCCTGAGCTGGTACAGTGACCCAGGCC
TGGCAGGCGTGTCCCTGACGGGGGGCCTGAGCTACAAAGAGGACACGAA
GGAGCTGGTGGTGGCCAAGGCTGGAGTCTACTATGTCTTCTTTCAACTA
GAGCTGCGGCGCGTGGTGGCCGGCGAGGGCTCAGGCTCCGTTTCACTTG
CGCTGCACCTGCAGCCACTGCGCTCTGCTGCTGGGGCCGCCGCCCTGGC
TTTGACCGTGGACCTGCCACCCGCCTCCTCCGAGGCTCGGAACTCGGCC
TTCGGTTTCCAGGGCCGCTTGCTGCACCTGAGTGCCGGCCAGCGCCTGG
GCGTCCATCTTCACACTGAGGCCAGGGCACGCCATGCCTGGCAGCTTAC
CCAGGGCGCCACAGTCTTGGGACTCTTCCGGGTGACCCCCGAAATCCCA
GCCGGACTCCCTTCACCGAGGTCGGAA[

In certain embodiments, the cell further comprises one exogenous co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_005182 (SEQ ID NO: 51) or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 51. SEQ ID NO: 51 is provided below.

[SEQ ID NO: 52]
MGHTRRQGTSPSKCPYLNFFQLLVLAGLSHFCSGVIHVTKEVKEVATLS
CGHNVSVEELAQTRIYWQKEKKMVLTMMSGDMNIWPEYKNRTIFDITNN
LSIVILALRPSDEGTYECVVLKYEKDAFKREHLAEVTLSVKADFPTPSI
SDFEIPTSNIRRIICSTSGGFPEPHLSWLENGEELNAINTTVSQDPETE
LYAVSSKLDFNMTTNHSFMCLIKYGHLRVNQTFNWNTTKQEHFPDNLLP
SWAITLISVNGIFVICCLTYCFAPRCRERRRNERLRRESVRPV

An exemplary nucleotide sequence encoding the amino acid sequence of SEQ ID NO: 51 is set forth in SEQ ID NO: 52. SEQ ID NO: 52 is provided below.

[SEQ ID NO: 52]
ATGGGCCACACACGGAGGCAGGGAACATCACCATCCAAGTGTCCATACC
TCAATTTCTTTCAGCTCTTGGTGCTGGCTGGTCTTTCTCACTTCTGTTC
AGGTGTTATCCACGTGACCAAGGAAGTGAAAGAAGTGGCAACGCTGTCC
TGTGGTCACAATGTTTCTGTTGAAGAGCTGGCACAAACTCGCATCTACT
GGCAAAAGGAGAAGAAAATGGTGCTGACTATGATGTCTGGGGACATGAA
TATATGGCCCGAGTACAAGAACCGGACCATCTTTGATATCACTAATAAC
CTCTCCATTGTGATCCTGGCTCTGCGCCCATCTGACGAGGGCACATACG
AGTGTGTTGTTCTGAAGTATGAAAAAGACGCTTTCAAGCGGGAACACCT
GGCTGAAGTGACGTTATCAGTCAAAGCTGACTTCCCTACACCTAGTATA
TCTGACTTTGAAATTCCAACTTCTAATATTAGAAGGATAATTTGCTCAA
CCTCTGGAGGTTTTCCAGAGCCTCACCTCTCCTGGTTGGAAAATGGAGA
AGAATTAAATGCCATCAACACAACAGTTTCCCAAGATCCTGAAACTGAG
CTCTATGCTGTTAGCAGCAAACTGGATTTCAATATGACAACCAACCACA
GCTTCATGTGTCTCATCAAGTATGGACATTTAAGAGTGAATCAGACCTT
CAACTGGAATACAACCAAGCAAGAGCATTTTCCTGATAACCTGCTCCCA
TCCTGGGCCATTACCTTAATCTCAGTAAATGGAATTTTTGTGATATGCT
GCCTGACCTACTGCTTTGCCCCAAGATGCAGAGAGAGAAGGAGGAATGA
GAGATTGAGAAGGGAAAGTGTACGCCCTGTA

In certain embodiments, the cell further comprises two exogenous co-stimulatory ligands that are 4-1BBL and CD80. In certain embodiments, the cell further comprises two exogenous co-stimulatory ligands that are 4-1BBL and CD80, wherein the 4-1BBL comprises or consists of the amino acid sequence set forth in SEQ ID NO: 49, and the CD80 comprises or consists of the amino acid sequence set forth in SEQ ID NO: 51.

Receptor-comprising cells comprising at least one exogenous co-stimulatory ligand are described in U.S. Pat. No. 8,389,282, which is incorporated by reference in its entirety.

### 5.3.3. Fusion Polypeptides

In certain embodiments, a presently disclosed cell comprising a presently disclosed antigen-recognizing receptor further comprises a fusion polypeptide. For example, a presently disclosed cell can be further transduced with the fusion polypeptide, such that the cell expresses or is induced to express the first antigen-recognizing receptor and the fusion polypeptide. The fusion polypeptide provides a co-stimulation signal to the cell. The fusion polypeptides are capable of enhancing the activity and/or efficacy of a cell comprising the first antigen-recognizing receptor (e.g., a CAR or a TCR like fusion molecule). In certain embodiments, the fusion polypeptide comprises a) an extracellular domain and a transmembrane domain of a co-stimulatory ligand, and b) an intracellular domain of a first co-stimulatory molecule.

Non-limiting examples of the co-stimulatory ligand include tumor necrosis factor (TNF) family members, immunoglobulin (Ig) superfamily members, and combinations thereof. The TNF family member can be selected from the group consisting of 4-1BBL, OX40L, CD70, GITRL, CD40L, and combinations thereof. The Ig superfamily member can be selected from the group consisting of CD80, CD86, ICOS ligand (ICOSLG (also known as “CD275”), and combinations thereof. In certain embodiments, the co-stimulatory ligand is selected from the group consisting of 4-1BBL, OX40L, CD70, GITRL, CD40L, CD80, CD86, ICOSLG, and combinations thereof.

In certain embodiments, the fusion polypeptide comprises an extracellular domain and a transmembrane domain of a co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 51 or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 51.

In certain embodiments, the extracellular domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 1-242 of SEQ ID NO: 51. In certain embodiments, the extracellular domain of CD80 comprises or consists of amino acids 1-242 of SEQ ID NO: 51 or a functional fragment thereof. A functional fragment can be a consecutive portion of amino acids 1-242 of SEQ ID NO: 51, which is at least about 50, at least about 75, at least about 100, at least about 125, at least about 150, at least about 175, or at least about 200, or at least about 220 amino acids in length. In certain embodiments, the functional fragment retains at least about 80%, at least about 85%, at least about 90%, at least about 95%, or at least about 100% of the primary function of the extracellular domain of CD80. Non-limiting examples of the primary functions of the extracellular domain of CD80 include binding to/interacting with CD28, binding to/interacting with CTLA-4, binding to/interacting with PD-L1, and contributing to CD80 homodimerization. In certain embodiments, an extracellular domain of CD80 comprises or consists of amino acids 1-242 of SEQ ID NO: 51).

In certain embodiments, the transmembrane domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 243-263 of SEQ ID NO: 51. In certain embodiments, the transmembrane domain of CD80 comprises or consists of amino acids 243-263 of SEQ ID NO: 51 or a fragment thereof. Such fragment can be at least about 5, at least about 10, at least about 15, or at least about 20 amino acids in length. In certain embodiments, the transmembrane domain of CD80 comprises or consists of amino acids 243-263 of SEQ ID NO: 51.

Non-limiting examples of co-stimulatory molecules include CD28, 4-1BB, OX40, ICOS, DAP-10, CD27, CD40, NKG2D, CD2, and combinations thereof.

In certain embodiments, the fusion polypeptide comprises an extracellular domain and a transmembrane domain of a co-stimulatory molecule that is 4-1BB. In certain embodiments, the co-stimulatory molecule is human 4-1BB. In certain embodiments, the 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 26 or a fragment thereof, and/or may optionally comprise up to one or up to two or up to three conservative amino acid substitutions. In certain embodiments, the 4-1BB comprises or consists of an amino acid sequence that is a consecutive portion of the amino acid sequence of SEQ ID NO: 30. In certain embodiments, the intracellular domain of 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 214-255 of SEQ ID NO: 26 or a fragment thereof, and/or may optionally comprise up to one or up to two or up to three conservative amino acid substitutions. In certain embodiments, the intracellular domain of 4-1BB comprises or consists of amino acids 214-255 of SEQ ID NO: 26 or a functional fragment thereof. Such functional fragment can be a consecutive portion of amino acids 214-255 of SEQ ID NO: 26, which is at least about 20, at least about 25, at least about 30, at least about 35, or at least about 40 amino acids in length. In certain embodiments, the functional fragment of amino acids 214-255 of SEQ ID NO: 26 retains at least about 80%, at least about 85%, at least about 90%, at least about 95%, or at least about 100% of the primary functions of the intracellular domain of 4-1BB. Non-limiting examples of the primary functions of the intracellular domain of 4-1BB include providing co-stimulatory signaling for the activation and proliferation of an immunoresponsive cell (e.g., a T cell), and interacting and activating downstream adaptors (e.g., TRAFs). In certain embodiments, the intracellular domain of 4-1BB comprises or consists of amino acids 214-255 of SEQ ID NO: 26.

In certain embodiments, the co-stimulatory molecule is CD28. In certain embodiments, the co-stimulatory molecule is human CD28. In certain embodiments, the CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 7 or a fragment thereof, and/or may optionally comprise up to one or up to two or up to three conservative amino acid substitutions. In certain embodiments, the CD28 comprises or consists of an amino acid sequence that is a consecutive portion of the amino acid sequence of SEQ ID NO: 7. In certain embodiments, the intracellular domain of CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 180 to 219 of SEQ ID NO: 7 or a fragment thereof, and/or may optionally comprise up to one or up to two or up to three conservative amino acid substitutions. In certain embodiments, the intracellular domain of CD28 comprises or consists of amino acids 180 to 219 of SEQ ID NO: 7 or a functional fragment thereof. A functional fragment of amino acids 180 to 219 of SEQ ID NO: 7 can be a consecutive portion of amino acids 180 to 219 of SEQ ID NO: 7, which is at least about 20, at least about 25, at least about 30, or at least about 35 amino acids in length. In certain embodiments, such functional fragment retains at least about 80%, at least about 85%, at least about 90%, at least about 95%, or at least about 100% of the primary function of the intracellular domain of CD28. Non-limiting examples of the primary functions of the intracellular domain of CD28 include providing co-stimulatory signaling for the activation and proliferation of an immunoresponsive cell (e.g., a T cell), and interacting with protein adaptors (e.g., PI3K, GRB2, and LCK). In certain embodiments, the intracellular domain of CD28 comprises or consists of amino acids 180 to 219 of SEQ ID NO: 7.

In certain embodiments, the fusion polypeptide comprises an intracellular domain of a second co-stimulatory molecule. In certain embodiments, the fusion polypeptide comprises an intracellular domain of a third co-stimulatory molecule. In certain embodiments, the fusion polypeptide comprises an intracellular domain of a fourth co-stimulatory molecule. In certain embodiments, the fusion polypeptide comprises an intracellular domain of a fifth co-stimulatory molecule. In certain embodiments, the first, second, third, fourth, and fifth co-stimulatory molecule can be the same or different among each other.

In certain embodiments, the fusion polypeptide comprises an extracellular domain and a transmembrane domain of a co-stimulatory ligand that is CD80, and an intracellular domain of a co-stimulatory molecule that is 4-1BB. In certain embodiments, the fusion polypeptide comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 53. In certain embodiments, the fusion polypeptide comprises or consists of the amino acid sequence set forth in SEQ ID NO: 53. SEQ ID NO: 53 is provided below.

[SEQ ID NO: 53]
MGHTRRQGTSPSKCPYLNFFQLLVLAGLSHFCSGVIHVTKEVKEVATLS
CGHNVSVEELAQTRIYWQKEKKMVLTMMSGDMNIWPEYKNRTIFDITNN
LSIVILALRPSDEGTYECVVLKYEKDAFKREHLAEVTLSVKADFPTPSI
SDFEIPTSNIRRIICSTSGGFPEPHLSWLENGEELNAINTTVSQDPETE
LYAVSSKLDFNMTTNHSFMCLIKYGHLRVNQTFNWNTTKQEHFPDNLLP
SWAITLISVNGIFVICCLTYCFKRGRKKLLYIFKQPFMRPVQTTQEEDG
CSCRFPEEEEGGCEL

In certain embodiments, the fusion polypeptide comprises an extracellular domain and a transmembrane domain of a co-stimulatory ligand that is CD80, an intracellular domain of a first co-stimulatory molecule that is 4-1BB, and an intracellular domain of a second co-stimulatory molecule that is CD28.

In certain embodiments, the fusion polypeptide comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 54. In certain embodiments, the fusion polypeptide comprises or consists of the amino acid sequence set forth in SEQ ID NO: 54. SEQ ID NO: 54 is provided below.

[SEQ ID NO: 54]
MGHTRRQGTSPSKCPYLNFFQLLVLAGLSHFCSGVIHVTKEVKEVATLS
CGHNVSVEELAQTRIYWQKEKKMVLTMMSGDMNIWPEYKNRTIFDITNN
LSIVILALRPSDEGTYECVVLKYEKDAFKREHLAEVTLSVKADFPTPSI
SDFEIPTSNIRRIICSTSGGFPEPHLSWLENGEELNAINTTVSQDPETE
LYAVSSKLDFNMTTNHSFMCLIKYGHLRVNQTFNWNTTKQEHFPDNLLP
SWAITLISVNGIFVICCLTYCFRSKRSRLLHSDYMNMTPRRPGPTRKHY
QPYAPPRDFAAYRKRGRKKLLYIFKQPFMRPVQTTQEEDGCSCRFPEEE
EGGCEL

Various modified fusion polypeptides are disclosed in International Patent Application No. PCT/US20/42753, which is incorporated by reference hereby in its entirety.

### 5.3.4. Gene Disruption of B2M Locus

In certain embodiments, a presently disclosed cell further comprises a gene disruption of a B2M locus. In certain embodiments, the gene disruption of the B2M locus results in a non-functional beta 2-microglobulin. In certain embodiments, the gene disruption of the B2M locus results in knockout of the B2M gene expression.

Any methods to generate the gene disruption as disclosed in Section 5.2.2.5 can be used to generate the gene disruption of the B2M locus. In certain embodiments, the gene disruption of the B2M locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the B2M locus can be a disruption of the coding region of the B2M locus and/or a disruption of the non-coding region of the B2M locus. In certain embodiments, the gene disruption of the B2M locus comprises a disruption of the coding region of the B2M locus. In certain embodiments, the gene disruption of the B2M locus comprises an insertion at the coding region of the B2M locus. Human B2M protein comprises four exons: exon 1, exon 2, exon 3, and exon 4. In certain embodiments, the coding region of the B2M locus comprises exon 1, exon 2, and exon 3. In certain embodiments, the gene disruption of the B2M locus comprises a disruption at one or both of exon 1 and exon 2 of the B2M locus. In certain embodiments, the gene disruption of the B2M locus comprises a disruption at exon 1 of the B2M locus. In certain embodiments, the gene disruption of the B2M locus comprises an insertion at exon 1 of the B2M locus.

Immune rejection of cells is due to the expression of human leucocyte antigen class I molecules (HLA-I) on the surface of these cells (Zhang et al., J Cell Mol Med. (2020);24:695-710). HLA-I presents “non-self” antigens to CD8⁺ T cells that eliminate the transplanted cells through direct cytotoxic effect (Zhang 2020). Due to the polymorphic nature of the HLA-I genes, it is often difficult to identify a perfect match between donor and recipient prior to transplantation (Zhang 2020). HLA-I comprises a heavy chain and a light chain, which is also called β₂-microglobulin (B2M). HLA-I structure is disrupted and non-functional when the B2M gene is deleted (Zhang 2020). In certain embodiments, the gene disruption of the B2M locus can reduce cell-induced immune rejection, thereby making the cells more suitable for an allogeneic setting.

### 5.3.5. Gene Disruption of CIITA Locus

In certain embodiments, a presently disclosed cell further comprises a gene disruption of a Class II transactivator (CIITA) locus. In certain embodiments, the gene disruption of the CIITA locus results in a non-functional MHC class II transactivator. In certain embodiments, the gene disruption of the CIITA locus results in knockout of the CIITA gene expression.

Any methods to generate the gene disruption as disclosed in Section 5.2.2.5 and any methods to generate the gene disruption of a B2M locus as disclose in 5.3.5 can be used to generate the gene disruption of the CIITA locus. In certain embodiments, the gene disruption of the CIITA locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the CIITA locus can be a disruption of the coding region of the CIITA locus and/or a disruption of the non-coding region of the CIITA locus. In certain embodiments, the gene disruption of the CIITA locus comprises a disruption of the coding region of the CIITA locus. In certain embodiments, the gene disruption of the CIITA locus comprises an insertion at the coding region of the CIITA locus. Human CIITA protein comprises 20 exons: exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, and exon 20. In certain embodiments, the coding region of the CIITA locus comprises exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, and exon 19. In certain embodiments, the gene disruption of the CIITA locus comprises a disruption at one or more of exon 1 through exon 19 of the CIITA locus. In certain embodiments, the gene disruption of the CIITA locus comprises a disruption at exon 3 of the CIITA locus. In certain embodiments, the gene disruption of the CIITA locus comprises an insertion at exon 3 of the CIITA locus.

CIITA is a transcriptional coactivator that regulates y-interferon-activated transcription of Major Histocompatibility Complex (MHC) class I and II genes (Devaiah et al., Frontiers in Immunology (2013); Vol. 4;Article 476:1-6). Thus, CIITA plays a critical role in immune responses: CIITA deficiency results in aberrant MHC gene expression and consequently in autoimmune diseases such as Type II bare lymphocyte syndrome (Devaiah 2013). Although CIITA does not bind to DNA directly, it regulates MHC transcription in two distinct ways—as a transcriptional activator and as a general transcription factor (Devaiah 2013). The CIITA is a master regulator of MHC gene expression (Devaiah 2013). CIITA induces de novo transcription of MHC class II genes and enhances constitutive MHC class I gene expression (Devaiah 2013). In certain embodiments, the gene disruption of the CIITA locus can reduce immune rejection, and improve survival of allogeneic immune cells, thereby making the cells more suitable for an allogeneic setting.

### 5.4. Formulations and Administration

The presently disclosed subject matter provides compositions comprising presently disclosed cells (e.g., disclosed in Section 5.3). In certain embodiments, the compositions are pharmaceutical compositions that further comprise a pharmaceutically acceptable excipient.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10⁵ cells will be administered, eventually reaching about 1× 10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. In certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus). The cells can be introduced by injection, catheter, or the like.

Compositions comprising the presently disclosed cells can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, compositions, or nucleic acid compositions are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells)) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 5.5. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor (or neoplasm) in a subject. The presently disclosed cells and compositions comprising thereof can be used for prolonging the survival of a subject suffering from a tumor. In certain embodiments, the tumor is cancer. The presently disclosed cells, compositions, and nucleic acid compositions can also be used for treating and/or preventing a pathogen infection or other infectious disease in a subject, such as an immunocompromised human subject. The presently disclosed cells, compositions, and nucleic acid compositions can also be used for treating and/or preventing an autoimmune disease in a subject. In certain embodiments, each of the above-noted method comprises administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of tumors (or neoplasms) include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, compositions, nucleic acid compositions can be used for treating and/or preventing blood cancers (e.g., leukemias, lymphomas, and myelomas) or ovarian cancer, which are not amenable to conventional therapeutic interventions.

In certain embodiments, the tumor and/or neoplasm is a solid tumor. Non limiting examples of solid tumor include renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the tumor and/or neoplasm is a blood cancer. Non-limiting examples of blood cancer include multiple myeloma, leukemia, and lymphomas. Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. The lymphoma can be Hodgkin's lymphoma or non-Hodgkin's lymphoma. In certain embodiments, the lymphoma is non-Hodgkin's lymphoma, including B-cell non-Hodgkin's lymphoma and T-cell non-Hodgkin's lymphoma.

In certain embodiments, the tumor and/or neoplasm is a B cell malignancy. Non-limiting examples of B cell malignancy include B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments, the tumor and/or neoplasm is a B cell-related neoplasm. Non-limiting examples of B cell-related neoplasm include chronic lymphocytic leukemia/small lymphocytic lymphoma (CLL/SLL), B-cell prolymphocytic leukemia, splenic marginal zone lymphoma, hairy cell leukemia, splenic B-cell lymphoma/leukemia (unclassifiable), splenic diffuse red pulp small B-cell lymphoma, lymphoplasmacytic lymphoma, Waldenström macroglobulinemia, monoclonal gammopathy of undetermined significance (MGUS, IgM), heavy-chain diseases (μ, γ, α), MGUS (IgG/A), plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with IRF4 rearrangement, primary cutaneous follicle center cell lymphoma, mantle cell lymphoma, in situ mantle cell neoplasia, diffuse large B-cell lymphoma (DLBCL) (not otherwise specified (NOS)), germinal center B-cell type, activated B-cell type, T-cell/histiocyte-rich large B-cell lymphoma, primary DLBCL of the central nervous system (CNS), primary cutaneous DLBCL (leg type), Epstein-Barr virus (EBV)-positive DLBCL (NOS), EBV-positive mucocutaneous ulcer, DLBCL associated with chronic inflammation, lymphomatoid granulomatosis, primary mediastinal (thymic) large B-cell lymphoma, intravascular large B-cell lymphoma, anaplastic lymphoma kinase (ALK)-positive large B-cell lymphoma, plasmablastic lymphoma, primary effusion lymphoma, human herpesvirus 8 (HHV-8)-associated DLBCL (NOS), Burkitt lymphoma, Burkitt-like lymphoma with 11q aberration, high-grade B-cell lymphoma with MYC and BLC2 and/or BCL6 rearrangements, high-grade B-cell lymphoma (NOS), and B-cell lymphoma (unclassifiable).

In certain embodiments, the tumor and/or neoplasm is a myeloid disorder. Non-limiting examples of myeloid disorders include myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

In certain embodiments, the myeloid disorder is acute myeloid leukemia (AML). In certain embodiments, the first and/or second antigens are independently selected from the group consisting of CD19, CD70, IL1RAP, ABCG2, AChR, ACKR6, ADAMTS13, ADGRE2, ADGRE2 (EMR2), ADORA3, ADRAID, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLECIA, CLL1, CNIH2, COL15A1, COLEC12, CPM, CR1, CX3CR1, CXCR4, CYP4F11, DAGLB, DARC, DFNB31, DGKI, EGFIR, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGRIA, FKBPIB, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LICAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNNID, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSFIB, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the myeloid disorder is acute myeloid leukemia (AML), and the first and/or second antigens are expressed on an AML hematopoietic stem/progenitor cell (HSPC) and/or a leukemia stem cell (LSC). In certain embodiments, the AML HSPC expresses CD34. In certain embodiments, the first antigen and/or the second antigen are not expressed or expressed at a non-detectable level in a non-hematopoietic stem cell and/or a non-hematopoietic progenitor cell. In certain embodiments, the first antigen and/or the second antigen are independently selected from the group consisting of CD70, IL1RAP, CD33, CLEC12A, ADGRE2, CD123, and combinations thereof.

The presently disclosed subject matter provides methods for treating and/or preventing a viral infection in a subject. The method can comprise administering an effective amount of the presently disclosed cells, a presently disclosed composition, or a presently disclosed nucleic acid composition to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

The presently disclosed subject matter provides methods for treating and/or preventing a bacterial infection in a subject. The method can comprise administering an effective amount of the presently disclosed cells, a presently disclosed composition, or a presently disclosed nucleic acid composition to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella, shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an autoimmune disease.

The presently disclosed subject matter provides methods for treating and/or preventing an infectious disease in a subject. The method can comprise administering an effective amount of the presently disclosed cells, a presently disclosed composition, or a presently disclosed nucleic acid composition to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed antigen-recognizing receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed antigen-recognizing receptor. The incorporation of a suicide gene into the presently disclosed antigen-recognizing receptor gives an added level of safety with the ability to eliminate the majority of receptor-expressing cells within a very short time period. A presently disclosed cell incorporated with a suicide gene can be pre-emptively eliminated at a given timepoint post the cell infusion or eradicated at the earliest signs of toxicity.

### 5.6. Kits

The presently disclosed subject matter provides kits for inducing and/or enhancing an immune response and/or treating and/or preventing a neoplasm or a pathogen infection (e.g., an autoimmune disease or an infectious disease) in a subject. In certain embodiments, the kit comprises compositions, buffers, nucleic acids, vectors, and reagents for producing the presently disclosed cells. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the cells, composition, or nucleic acid composition are provided together with instructions for administering the cells, composition, or nucleic acid composition to a subject having or at risk of developing a tumor (e.g., a cancer) or a pathogen infection (e.g., an infectious disease), or immune disorder (e.g., an autoimmune disease). The instructions generally include information about the use of the cell, composition or nucleic acid composition for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.7. Exemplary Embodiments

Clause 1. A method of producing an immunoresponsive cell comprising an antigen-recognizing receptor, the method comprising: a) positively selecting CD3⁺ immunoresponsive cells solely with a CD3⁺ monospecific reagent; b) obtaining a composition enriched for CD3⁺ immunoresponsive cells; c) contacting the CD3⁺ immunoresponsive cells with a reagent activating and/or stimulating the immunoresponsive cells; and d) introducing an antigen-recognizing receptor into the immunoresponsive cells.

Clause 2. The method of clause 1, wherein the CD3⁺ monospecific reagent is an anti-CD3 antibody or an antigen-binding fragment thereof.

Clause 3. The method of clause 2, wherein the anti-CD3 antibody or an antigen-binding fragment thereof does not activate the immunoresponsive cell.

Clause 4. The method of clause 2 or 3, wherein the anti-CD3 antibody or an antigen-binding fragment thereof binds to an epitope located on the human CD3ε chain.

Clause 5. The method of any one of clauses 2-4, wherein the anti-CD3 antibody or antigen-fragment thereof is bound to a magnetic particle.

Clause 6. The method of any one of clauses 2-4, wherein the anti-CD3 antibody or antigen-fragment thereof is bound to an iron-dextran particle.

Clause 7. The method of clause 5 or 6, wherein the magnetic particle has a diameter of from about 10 nm to about 100 nm.

Clause 8. The method of clause 7, wherein the magnetic particle has a diameter of about 50 nm.

Clause 9. The method of any one of clauses 2-8, wherein CD3⁺ monospecific reagent is contacted with a sample at a cell density of about 100×10⁶ cells/ml.

Clause 10. The method of any one of clauses 1-9, wherein the positive selection is a chromatography-based selection.

Clause 11. The method of any one of clauses 1-10, wherein the composition comprises at least about 80%, at least about 85%, at least about 90%, at least about 95%, at least about 96%, at least about 97%, at least about 98%, at least about 99%, or about 100% more CD3⁺ immunoresponsive cells compared to a sample.

Clause 12. The method of clause 11, wherein the composition comprises at least about 90% more CD3⁺ immunoresponsive cells compared to the sample.

Clause 13. The method of any one of clauses 1-12, wherein the composition comprises at least about 60%, at least about 65%, at least about 70%, at least about 75%, at least about 80%, at least about 85%, at least about 90%, at least about 95%, at least about 96%, at least about 97%, at least about 98%, at least about 99%, or about 100% of CD3⁺ immunoresponsive cells over the total number of cells.

Clause 14. The method of clause 13, wherein the composition comprises at least about 90% of CD3⁺ immunoresponsive cells over the total number of cells.

Clause 15. The method of any one of clauses 1-14, wherein the composition comprises at least about 80%, at least about 85%, at least about 90%, at least about 95%, at least about 96%, at least about 97%, at least about 98%, at least about 99%, or about 100% less CD14⁺ immunoresponsive cells compared to a sample.

Clause 16. The method of clause 15, wherein the composition comprises at least about 80% less CD14⁺ immunoresponsive cells compared to the sample.

Clause 17. The method of any one of clauses 1-16, wherein the composition comprises at least about 1%, at least about 5%, at least about 10%, at least about 15%, or at least about 20% of CD14⁺ immunoresponsive cells over the total number of cells.

Clause 18. The method of clause 17, wherein the composition comprises at least about 5% of CD14⁺ immunoresponsive cells over the total number of cells.

Clause 19. The method of any one of clauses 1-18, wherein the reagent activating and/or stimulating the immunoresponsive cells comprises an anti-CD3 antibody or an antigen-binding fragment thereof activating the immunoresponsive cell.

Clause 20. The method of any one of clauses 1-19, wherein the reagent activating and/or stimulating the immunoresponsive cells comprises an anti-CD28 antibody or an antigen-binding thereof.

Clause 21. The method of any one of clauses 1-20, wherein the reagent activating and/or stimulating the immunoresponsive cells comprises an anti-CD3 antibody or an antigen-binding fragment thereof activating the immunoresponsive cell and an anti-CD28 antibody or an antigen-binding thereof.

Clause 22. The method of any one of clauses 19-21, wherein the reagent is bound to a magnetic particle.

Clause 23. The method of any one of clauses 1-22, wherein the immunoresponsive cells are T cells.

Clause 24. The method of clause 23, wherein the T cells are selected from the group consisting of cytotoxic T lymphocytes (CTL), γδ T cells, tumor-infiltrating lymphocytes (TIL), regulatory T cells, and Natural Killer T (NKT) cells.

Clause 25. The method of clause 23 or 24, wherein the T cells are CD8⁺ T cells.

Clause 26. The method of clause 23 or 24, wherein the T cells are CD4⁺ T cells.

Clause 27. The method of clause 25, wherein the CD8⁺ T cells are CD4 independent.

Clause 28. The method of any one of clauses 1-27, wherein the immunoresponsive cell are CD14⁻ cells.

Clause 29. The method of any one of clauses 1-28, wherein the immunoresponsive cells are obtained from a sample selected from the group consisting of a blood sample, a blood-derived sample, an apheresis product, a leukapheresis product, a whole blood sample, a peripheral blood mononuclear cells (PBMCs) sample, a leukocytes sample, a bone marrow sample, a thymus sample, a tissue biopsy sample, a tumor sample, a leukemia sample, a lymphoma sample, a lymph node sample, a gut associated lymphoid tissue sample, a mucosa associated lymphoid tissue sample, a spleen sample, a liver sample, a lung sample, a stomach sample, an intestine sample, a colon sample, a kidney sample, a pancreas sample, a breast sample, a bone sample, a prostate sample, a cervix sample, a testes sample, an ovaries sample, a tonsil sample, and a combination thereof.

Clause 30. The method of clause 29, wherein the sample is an apheresis product.

Clause 31. The method of clause 29, wherein the sample is a leukapheresis product.

Clause 32. The method of any one of clauses 1-31, wherein the immunoresponsive cells are obtained from a sample of a subject having acute myeloid leukemia (AML).

Clause 33. The method of any one of clauses 1-32 further comprising a density-based cell separation.

Clause 34. The method of 33, wherein the density-based cell separation allows purification of peripheral blood mononuclear cells (PBMCs).

Clause 35. The method of any one of clauses 1-34, wherein the antigen-recognizing receptor targets an antigen.

Clause 36. The method of clause 35, wherein the antigen is a tumor antigen or a pathogen antigen.

Clause 37. The method of clause 36, wherein the tumor antigen is selected from the group consisting of CD19, CD70, IL1RAP, ABCG2, AChR, ACKR6, ADAMTS13, ADGRE2 (EMR2), ADORA3, ADRAID, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLECIA, CLL1, CNIH2, COL15A1, COLEC12, CPM, CR1, CX3CR1, CXCR4, CYP4F11, DAGLB, DARC, DFNB31, DGKI, EGFIR, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGRIA, FKBPIB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL1ORB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LICAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNNID, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

Clause 38. The method of any one of clauses 1-37, wherein the antigen-recognizing receptor is a chimeric receptor, a T cell receptor (TCR), or a TCR like fusion molecule.

Clause 39. The method of clause 38, wherein the antigen-recognizing receptor is a chimeric receptor.

Clause 40. The method of clause 39, wherein the chimeric receptor is a chimeric antigen receptor (CAR).

Clause 41. The method of clause 40, wherein the CAR comprises an extracellular antigen-binding domain that binds to the antigen, and an intracellular signaling domain that is capable of delivering an activation signal to the cell.

Clause 42. The method of clause 41, wherein the intracellular signaling domain of the CAR comprises a CD3% polypeptide.

Clause 43. The method of clause 42, wherein the CD3ζ polypeptide is a native CD3ζ polypeptide or a modified CD3ζ polypeptide.

Clause 44. The method of clause 43, wherein the modified CD3ζ polypeptide comprises a native ITAM1, an ITAM2 variant consisting of two loss-of-function mutations, and an ITAM3 variant consisting of two loss-of-function mutations.

Clause 45. The method of any one of clauses 41-44, wherein the intracellular signaling domain of the CAR further comprises at least one costimulatory signaling region.

Clause 46. The method of clause 45, wherein the at least one costimulatory signaling region comprises at least an intracellular domain of a co-stimulatory molecule or a portion thereof.

Clause 47. The method of clause 46, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

Clause 48. The method of any one of clauses 41-47, wherein the CAR comprises a transmembrane domain.

Clause 49. The method of clause 39, wherein the chimeric receptor is a chimeric co-stimulating receptor (CCR).

Clause 50. The method of clause 49, wherein the CCR comprises an extracellular antigen-binding domain that binds to the second antigen and an intracellular domain that is capable of delivering a costimulatory signal to the cell but does not alone deliver an activation signal to the cell.

Clause 51. The method of clause 50, wherein the intracellular domain of the CCR comprises at least an intracellular domain of a co-stimulatory molecule or a portion thereof.

Clause 52. The method of clause 51, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

Clause 53. The method of clause 38, wherein the antigen-recognizing receptor is a TCR like fusion molecule.

Clause 54. The method of clause 53, wherein the TCR like fusion molecule comprises i) a first antigen-binding chain comprising an antigen-binding fragment of a heavy chain variable region (V_{H}) of an antibody; and ii) a second antigen-binding chain comprising an antigen-binding fragment of a light chain variable region (V_{L}) of the antibody; wherein the first and second antigen-binding chains a) each comprise a TRAC polypeptide or a TRBC polypeptide, and b) bind to the first antigen, wherein the TCR like fusion molecule binds to the first antigen in an HLA-independent manner.

Clause 55. The method of clause 54, wherein at least one of the TRAC polypeptide and the TRBC polypeptide is endogenous.

Clause 56. The method of clause 54 or 55, wherein the antigen-binding chain comprises an antigen-binding fragment of a V_{H} of an antibody and a TRBC polypeptide, and the second antigen-binding chain comprises an antigen-binding fragment of a V_{L} of the antibody and a TRAC polypeptide.

Clause 57. The method of any one of clauses 54-56, wherein the antigen-binding chain comprises an antigen-binding fragment of a V_{H} of an antibody and a TRAC polypeptide, and the second antigen-binding chain comprises an antigen-binding fragment of a V_{L} of the antibody and a TRBC polypeptide.

Clause 58. The method of any one of clauses 54-57, wherein the first and second antigen binding chains are capable of associating with a CD3ζ polypeptide.

Clause 59. The method of clause 58, wherein the first and second antigen binding chains, upon binding to the first antigen, are capable of activating the CD3ζ polypeptide.

Clause 60. A composition comprising the immunoresponsive cell obtained by the method of any one of clauses 1-59.

Clause 61. The composition of clause 60, which is a pharmaceutical composition further comprising a pharmaceutically acceptable excipient.

Clause 62. A method of reducing tumor burden in a subject, the method comprising administering to the subject an effective amount of the immunoresponsive cell produced by the method of any one of clauses 1-59 or the composition of claim 60 or 61.

Clause 63. The method of clause 62, wherein the method reduces the number of tumor cells, reduces tumor size, and/or eradicates the tumor in the subject.

Clause 64. A method of preventing and/or treating a neoplasm or a tumor in the subject, administering to the subject an effective amount of the immunoresponsive cell produced by the method of any one of clauses 1-59 or the composition of claim 60 or 61.

Clause 65. The method of any one of clauses 62-64, wherein the neoplasm or tumor is cancer.

Clause 66. The method of any one of clauses 62-65, wherein the neoplasm or tumor is a solid tumor.

Clause 67. The method of any one of clauses 62-65, wherein the neoplasm or tumor is a blood cancer.

Clause 68. The method of any one of clauses 62-65, and 67, wherein the neoplasm or tumor is a myeloid disorder.

Clause 69. The method of clause 68, wherein the myeloid disorder is selected from the group consisting of myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

Clause 70. The method of clause 69, wherein the myeloid disorder is acute myeloid leukemia (AML).

Clause 71. A method of preventing and/or treating a pathogen infection in a subject, the method comprising administering to the subject an effective amount of the immunoresponsive cell produced by the method of any one of clauses 1-59 or the composition of claim 60 or 61.

Clause 72. A method of preventing and/or treating an autoimmune disease in a subject, the method comprising administering to the subject an effective amount of the immunoresponsive cell produced by the method of any one of clauses 1-59 or the composition of claim 60 or 61.

Clause 73. A method of preventing and/or treating an infectious disease in a subject, the method comprising administering to the subject an effective amount of the immunoresponsive cell produced by the method of any one of clauses 1-59 or the composition of claim 60 or 61.

Clause 74. A kit comprising the immunoresponsive cell produced by the method of any one of clauses 1-59 or the composition of claim 60 or 61.

Clause 75. The kit of clause 74, wherein the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.

Clause 76. The composition of clause 60 or 61 or the kit of claim 74 or 75 for use in reducing tumor burden, treating and/or preventing a neoplasm or a tumor, preventing and/or treating a pathogen infection, preventing and/or treating an autoimmune disease, and/or preventing and/or treating an infectious disease, in a subject.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### EXAMPLE 1

Traditional CAR-T cell manufacturing methods use anti-CD4/CD8 beads (e.g., Miltenyi microbeads) or anti-CD3/CD28 beads (e.g., Dynabeads) for the selection of CD3⁺ T cells because the use of sole anti-CD3 antibodies can mediate tolerance mechanisms and induce anergy of the T cells (Andris et al., J Immunol. 2004 Sep. 1;173 (5): 3201-8). Indeed, anti-CD3/CD28 beads have proved a convenient reagent for expansion of T cells. In contrast to such traditional approaches, the subject matter of the present disclosure relates to positive selection of CD3⁺ T cells, e.g., using anti-CD3 microbeads. Such an approach runs counter to the general assumption that such anti-CD3 microbeads would lead to anergyzing T cells, and thus making the T cells less cytotoxic and less potent.

### Small-Scale T Cell Selection Using CD3⁺ Microbeads

Healthy donor or patient PBMCs were washed twice with CliniMACS PBS/EDTA and 0.5% HSA selection buffer and resuspended in the same buffer at a density of 125×10⁶ or 16⁷×10⁶ viable cells/mL. Cells were then incubated with injectable immune globin (IVIG) at a final concentration of 20 mg/mL IVIG and 100×10⁶ cells/mL for 5 min at room temperature. 20 μL or 40 μL of CD3 microbeads (Miltenyi, cat #130-050-101) were subsequently added as per every 10×10⁶ total viable cells to obtain an incubation cell density of 100×10⁶ cells/mL. Cells and beads mixture was put on a rocker and further incubated for 30 minutes with gentle rocking at room temperature. Upon completing bead incubation, cells were washed again with CliniMACS PBS/EDTA buffer+0.5% HSA buffer to remove unbound beads. CD3 microbead-bound cells were resuspended to 200×10⁶ cells/mL in the same buffer and placed in a pre-wet LS columns (Miltenyi, cat #130-042-401) on MidiMACS™ Separator magnet. Negative fraction flow through was collected and the column was washed three times with the same selection buffer further remove impurity. The LS columns were then removed from the MidiMACS™ Separator magnet. 5 mL of selection buffer was added, and positive faction of the cells were pushed out from the column using the plunger and collected for downstream process. Additional information on the CD3 microbeads can be found in Table 1 below:

TABLE 1
Dynabeads CD3/CD28 vs Miltenyi CD3 Microbeads

| | | Miltenyi |
|---|---|---|
| | Dynabeads CD3/CD28 | Microbeads |
| Size | Diameter of | 50 nm |
| | 4.5 mM +/− 5% CV | |
| Type | Paramagnetic, | Nanobeads |
| | polymer coated | |
| | polystyrene particles | |
| Uniformity | Spherical, 1-3% | None |
| | variability | |
| T-Cell Release | Yes | No |
| Columns Required | No | Yes |
| Internalized into | No | Yes |
| T-Cells | | |
| Retained in T-Cells | No | Yes |
| after 72 hrs | | |

### Large-Scale T Cell Selection Using CD3 GMP Microbeads

Healthy donor or patient apheresis product was first washed with a cell washer (such as the LOVO cell processing system) to remove platelet and reduce red blood cell contamination. 7.5 mL of injectable immune globin (IVIG) was added to post-wash cellular product, and incubated with cells for 5 minutes with gentle rocking at room temperature. 7.5 mL of CliniMACS® CD3 GMP MicroBeads (cat #170-076-712) were added afterwards, and the washed cellular product, IVIG and CliniMACS® CD3 GMP MicroBeads were incubated for an additional 30 minutes at room temperature with gentle rocking. Once the incubation was completed, unbound beads were washed away using a cell washer, such as the LOVO cell processing system. CD3 bead-bound cells were subsequently selected using CliniMACS Plus system with Enrichment 1.1 program and CliniMACS PBS/EDTA+0.5% HSA selection buffer.

### T Cell Selection Using CTS™ Dynabeads™ CD3/CD28

Healthy donor or patient apheresis product was first washed with a cell washer (such as the LOVO cell processing system). Washed cells were subsequently incubated for 1 hour with agitation at room temperature with CTS™ Dynabeads™ CD3/CD28 (ThermoFisher) at beads to T cell ratio of 3:1. Following incubation cells were bound and enriched using a Dynal ClinEx VIVO™ MPC magnet. CD3⁺ enriched cells were resuspended in culture and transferred to a 37° C. incubator for a desired length of activation before the downstream procedure such as transduction. Additional information on the CTS™ Dynabeads™ CD3/CD28 can be found in Table 1.

Small-Scale T Cell Selection Using CD4⁺/CD8⁺ Microbeads

Healthy donor or patient PBMCs were washed twice with CliniMACS PBS/EDTA+0.5% HSA selection buffer and resuspended in the same buffer at 16⁷×10⁶ or 500×10⁶ viable cells/mL. 20 μL of each CD4⁺ microbeads (Miltenyi, cat #170-076-702) and CD8⁺ microbeads (Miltenyi, cat #170-076-703), or 40 μL of each CD4⁺ microbeads and CD8⁺ were subsequently added as per every 10×10⁶ total viable cells to obtain an incubation cell density of 100× 10⁶ cells/mL. Cells and beads mixture was put on a rocker and further incubated for 30 minutes with gentle rocking at room temperature. Upon bead incubation was complete, cells were washed again with CliniMACS PBS/EDTA buffer+0.5% HSA buffer to remove unbound beads. CD4 and CD8 microbead-bound cells were resuspended to 200×10⁶/mL in the same buffer and placed in a pre-wet LS columns (Miltenyi, cat #130-042-401) on MidiMACS™ Separator magnet. Negative fraction flow through was collected and column was washed three times with the same selection buffer to further remove impurity. The LS columns were then removed from the MidiMACS™ Separator magnet. 5 mL of selection buffer was added and positive faction of the cells were pushed out from the column using the plunger and collected for downstream process.

### Large-Scale T Cell Selection Using CD4+&CD8+ GMP Microbeads

Healthy donor or patient apheresis product was first washed with a cell washer, such as the LOVO cell processing system, to remove platelet and reduce red blood cell contamination. 7.5 mL of CliniMACS® CD4⁺ GMP Microbeads (Miltenyi, cat #170-076-702) and 7.5 mL of CD8⁺ GMP Microbeads (Miltenyi, cat #170-076-703) were subsequently added to the cells. The washed cellular products, CliniMACS® CD4 GMP MicroBeads, and CliniMACS® CD8 GMP MicroBeads were incubated for an additional 30 minutes at room temperature with gentle rocking. Once the incubation was completed, unbound beads were washed away using a cell washer, such as the LOVO cell processing system. CD4⁺ Microbead-bound cells and CD8⁺ microbeads-bound cells were subsequently selected using CliniMACS Plus system with Enrichment 1.1 program and CliniMACS PBS/EDTA+0.5% HSA selection buffer.

Evaluation of the amount of CD14⁺ cells isolated using the CD3⁺ selection

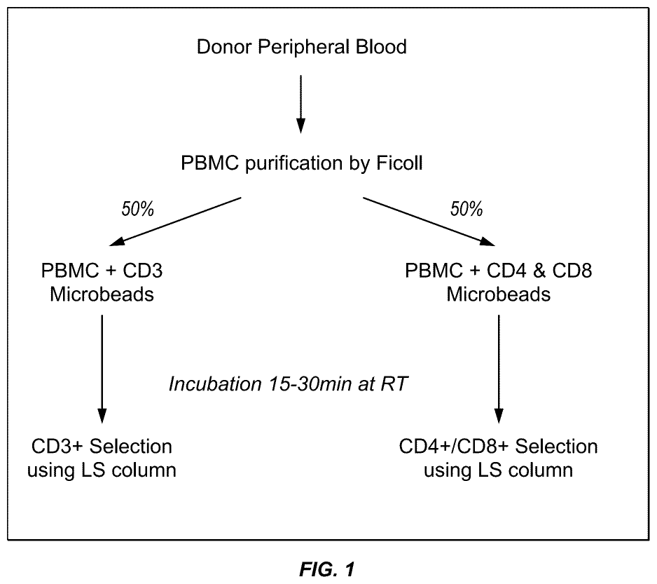

Healthy donor or patient donor PBMCs were split equally, half of the cells were selected with either CD3⁺ selection method and the other half of the cells were selected with the CD4⁺/CD8⁺ selection method as described above (FIG. 1). Pre- and post-selection cell compositions for CD3⁺, CD4⁺, CD8⁺, CD14⁺, CD33⁺ and CD34⁺ were evaluated by FACS analysis and summarized in the table below. As shown in Table 2, the T cell purity was significantly higher using the CD3⁺ selection method compared to the CD4⁺/CD8⁺ selection method (p=0.0005 by paired two-tail student t-test).

TABLE 2
Pre- and post-selection product cellular composition

| | | CD3+ | CD4+ | CD8+ | CD14+ | CD33+ | CD34+ |
|---|---|---|---|---|---|---|---|
| Donor | Selection Method | (%) | (%) | (%) | (%) | (%) | (%) |
| #1 | Pre-Selection | 57.4 | 35.7 | 19.5 | 17.1 | 19.7 | 0.0 |
| | Post-CD3 + Selection | 98.9 | 60.5 | 36.6 | 0.5 | 0.5 | 0.0 |
| | Post-CD4 + CD8 + Selection | 72.8 | 52.4 | 19.4 | 17.02 | 18.4 | 0.0 |
| #2 | Pre-Selection | 25.1 | 17.5 | 2.5 | 10.1 | 48.3 | 15.4 |
| | Post-CD3 + Selection | 87.2 | 71.2 | 19.0 | 8.2 | 7.1 | 0.6 |
| | Post-CD4 + CD8 + Selection | 34.4 | 26.3 | 2.4 | 58 | 51.9 | 2.6 |
| #3 | Pre-Selection | 0.6 | 0.1 | 0.5 | 0.5 | 22.7 | 97.8 |
| | Post-CD3 + Selection | 87.3 | 11.4 | 81.3 | 3.4 | 5.6 | 10.5 |
| | Post-CD4 + CD8 + Selection | 4.7 | 0.2 | 1.1 | 19.7 | 21.3 | 33.9 |
| #4 | Pre-Selection | 7.42 | 4.5 | 2.3 | 5.1 | 73.7 | 8.4 |
| | Post-CD3 + Selection | 90.2 | 50.5 | 38 | 5.2 | 1 | 3.7 |
| | Post-CD4 + CD8 + Selection | 36.1 | 19.5 | 10.7 | 7 | 12.7 | 15.8 |
| #5 | Pre-Selection | 71.0 | 53.8 | 20.5 | 5.5 | 2.2 | 0.1 |
| | Post-CD3 + Selection | 99.2 | 65.0 | 30.6 | 0.5 | 0.1 | 0.0 |
| | Post-CD4 + CD8 + Selection | 60.1 | 76.9 | 10.5 | 28.3 | 0.0 | 0.0 |
| #6 | Pre-Selection | 57.6 | 44.7 | 22.5 | 6.5 | 1.6 | 0.4 |
| | Post-CD3 + Selection | 98.6 | 61.0 | 35.3 | 0.9 | 0.1 | 0.1 |
| | Post-CD4 + CD8 + Selection | 50.5 | 67.2 | 9.1 | 25.7 | 0.1 | 0.4 |
| #7 | Pre-Selection | 12.0 | 5.5 | 6.1 | 31.6 | 14.1 | 40.8 |
| | Post-CD3 + Selection | 92.3 | 43.2 | 58.2 | 18.1 | 2.7 | 10.5 |
| | Post-CD4 + CD8 + Selection | 59.5 | 30.4 | 28.5 | 30.7 | 22.6 | 1.1 |

Donors #1, 5 and 6 are heathy donors; and Donors #2, 3, 4 and 7 are AML patient donors.

### Evaluation of the Amount of CD14⁺ Cells Isolated Using Dynabeads CD3/CD28 Selection

CD14⁺ cells were not measured immediately post-Dynabeads CD3/CD28 selection because of T cells binding tightly to Dynabeads after the selection. A recent group reported that Dynabeads CD3/CD28 were engulfed by CD14⁺ monocytes and these CD14⁺ cells were subsequently pulled down by the MPC magnet (Wang et al., Mol Ther Methods Clin Dev. 2021 Jul. 16; 22:377-387). These CD14⁺ monocytes contamination inherent to the Dynabeads CD3/CD28 selection method are a challenge for CAR-T cell manufacturing.

Evaluation of the Functionality of CAR-T Cells Generated from CD3⁺ or CD4⁺/CD8⁺ Selected Cells

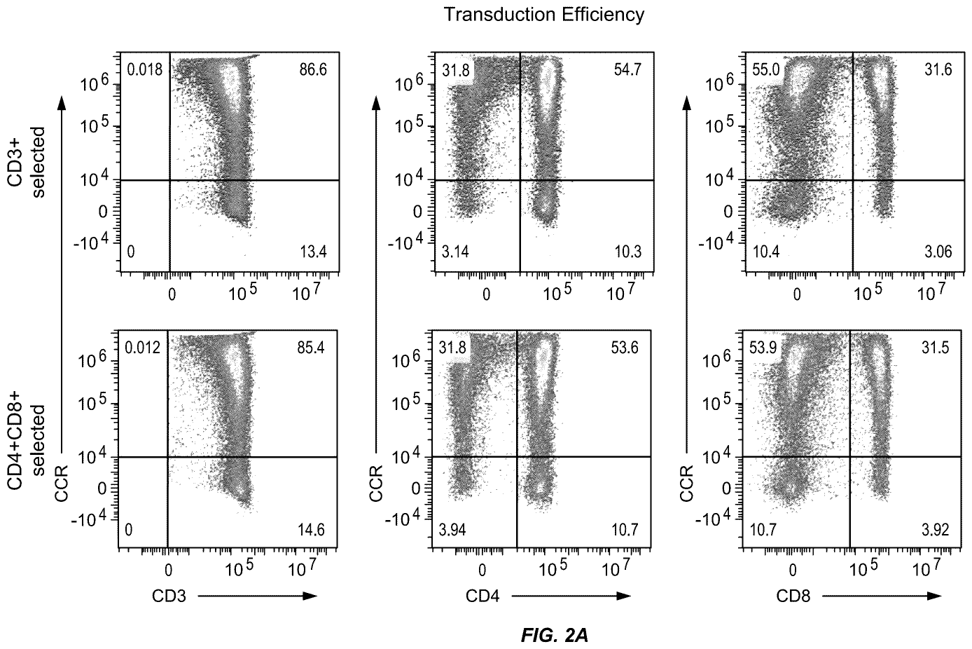

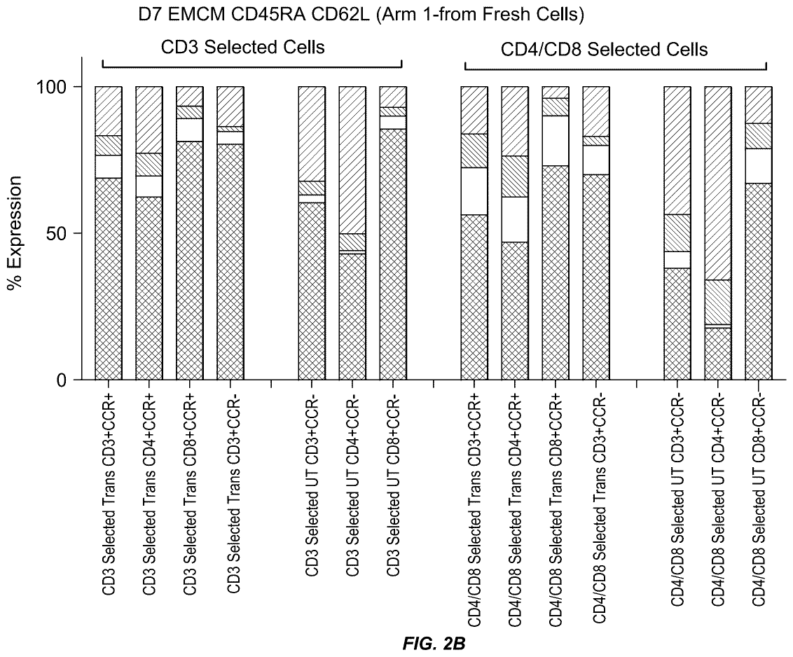

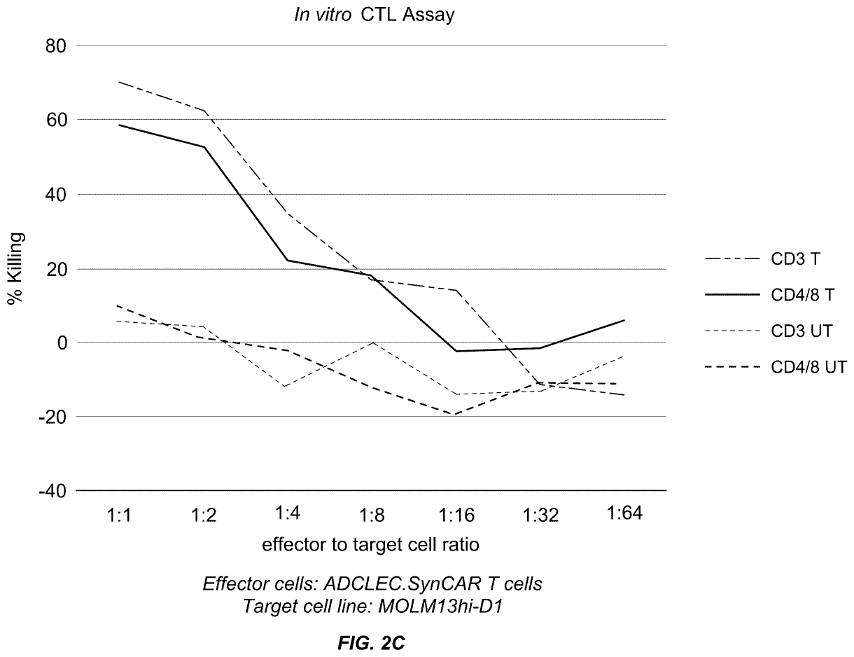

Since the CD3⁺ selection method produced higher purity cell populations, it was determined whether CAR-T cells generated using the CD3⁺ selection method had improved functionality. Briefly, cells purified using CD3⁺ selection method or the CD4⁺/CD8⁺ selection method outlined above were transduced with a combinatorial CAR-CCR vector, termed ADCLEC.syn, which encodes a CAR specific for ADGRE2 and a CCR specific for CLEC12A to treat R/R AML. Additional information on the ADCLEC.syn can be found in International Patent Application No. PCT/US2022/026131. As shown in FIG. 2A, cells purified using the CD3⁺ selection method had comparable transduction efficiency in the test subtype of T cells (e.g., CD3⁺, CD4⁺, and CD8⁺). Further, these cells showed comparable memory cell EM/CM phenotype (FIG. 2B). Finally, these CAR-T cells were incubated with the AML cell line MOLM13 hi-D1. As shown in FIG. 2C, the CAR-T cells generated using the CD3⁺ selection method or the CD4⁺/CD8⁺ selection method had comparable in vitro killing activity. Overall, these data demonstrate that the CD3⁺ selection method disclosed herein produces functional CAR-T cells.

Evaluation of the In Vivo Antitumor Activity of CAR-T Cells Generated from CD3⁺ or CD4⁺/CD8⁺ Selected Cells

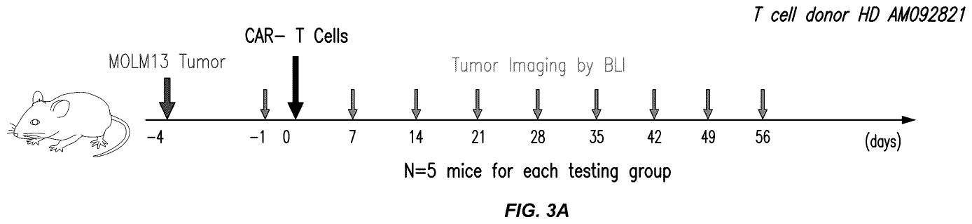

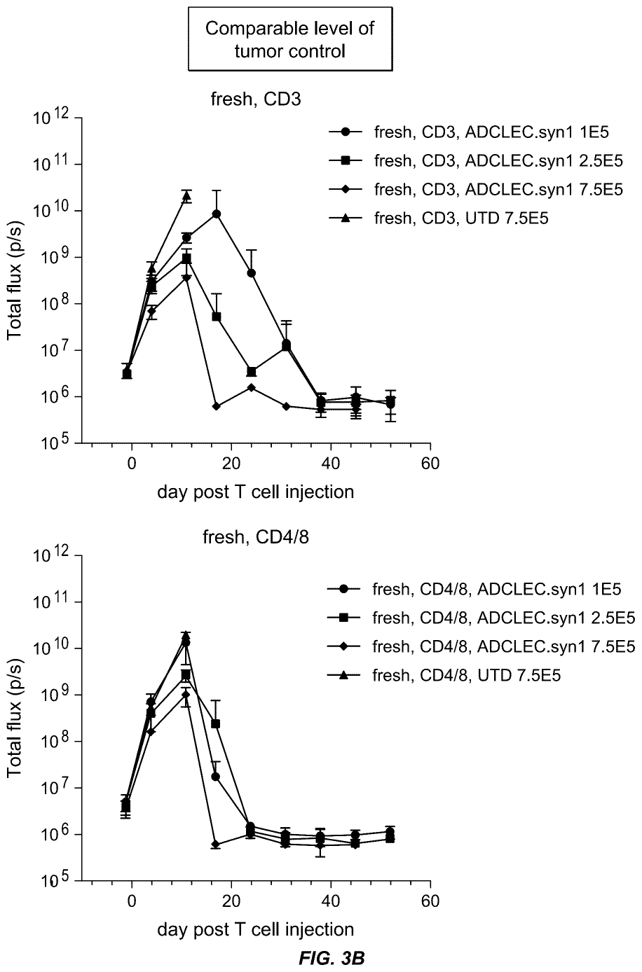

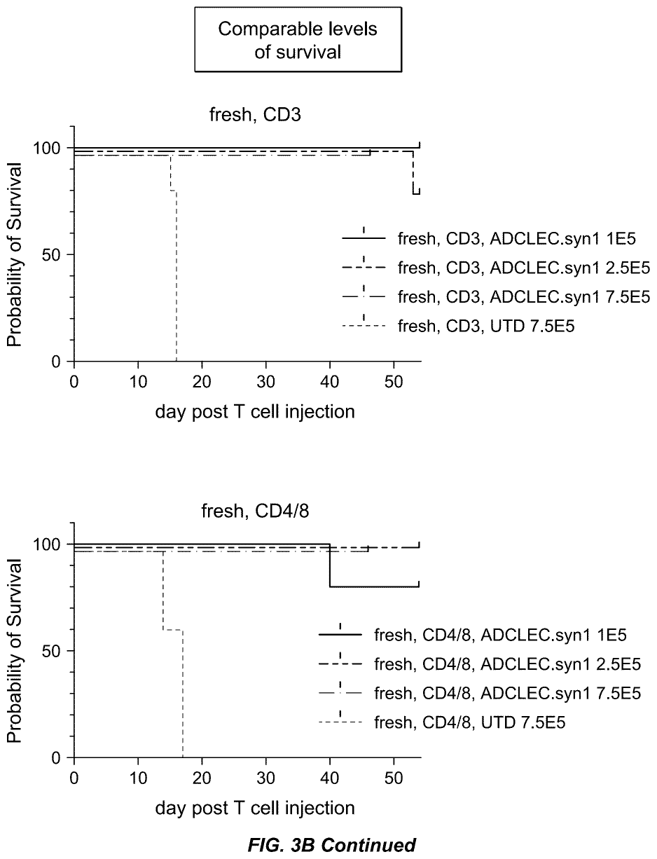

Next, the in vivo activity of these cells was tested. Briefly, tumors were initially established in mice which then received different doses of CAR-T cells or untransduced T cells (FIG. 3A). Tumor imaging was assessed by BLI and survival curves were also determined. As shown in FIG. 3B, CAR-T cells obtained from the CD3⁺ selection method or the CD4⁺/CD8⁺ selection method has comparable levels of tumor control and of survival. Overall, these data demonstrate that the CD3⁺ selection method disclosed herein produces functional CAR-T cells that achieve at least comparable levels of in vivo killing.

### CONCLUSIONS

In the present example, it is demonstrated that the positively selected CD3⁺ T cells can be used to manufacture potent CAR T cells. Moreover, the present example shows that this process is efficient at removing AML tumor cells, making this method particularly useful for specific applications (e.g., the production of CAR-T cells for AML).

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of producing an immunoresponsive cell comprising an antigen-recognizing receptor, the method comprising:
a) positively selecting CD3⁺ immunoresponsive cells solely with a CD3⁺ monospecific reagent; b) obtaining a composition enriched for CD3⁺ immunoresponsive cells;
c) contacting the CD3⁺ immunoresponsive cells with a reagent activating and/or stimulating the immunoresponsive cells; and
d) introducing an antigen-recognizing receptor into the immunoresponsive cells.

2. The method of claim 1, wherein the CD3⁺ monospecific reagent is an anti-CD3 antibody or an antigen-binding fragment thereof.

3. The method of claim 2, wherein the anti-CD3 antibody or an antigen-binding fragment thereof does not activate the immunoresponsive cell.

4. The method of claim 2 or 3, wherein the anti-CD3 antibody or an antigen-binding fragment thereof binds to an epitope located on the human CD3F chain.

5. The method of claim 2, wherein the anti-CD3 antibody or antigen-fragment thereof is bound to a magnetic particle.

6. The method of claim 2, wherein the anti-CD3 antibody or antigen-fragment thereof is bound to an iron-dextran particle.

7. The method of claim 5, wherein the magnetic particle has a diameter of from about 10 nm to about 100 nm.

8. The method of claim 7, wherein the magnetic particle has a diameter of about 50 nm.

9. The method of claim 1, wherein CD3⁺ monospecific reagent is contacted with a sample at a cell density of about 100×10⁶ cells/ml.

10. The method of claim 1, wherein the positive selection is a chromatography-based selection.

11. The method of claim 1, wherein the composition comprises at least about 80%, at least about 85%, at least about 90%, at least about 95%, at least about 96%, at least about 97%, at least about 98%, at least about 99%, or about 100% more CD3⁺ immunoresponsive cells compared to a sample.

12. The method of claim 11, wherein the composition comprises at least about 90% more CD3⁺ immunoresponsive cells compared to the sample.

13. The method of claim 1, wherein the composition comprises at least about 60%, at least about 65%, at least about 70%, at least about 75%, at least about 80%, at least about 85%, at least about 90%, at least about 95%, at least about 96%, at least about 97%, at least about 98%, at least about 99%, or about 100% of CD3⁺ immunoresponsive cells over the total number of cells.

14. The method of claim 13, wherein the composition comprises at least about 90% of CD3⁺ immunoresponsive cells over the total number of cells.

15. The method of claim 1, wherein the composition comprises at least about 80%, at least about 85%, at least about 90%, at least about 95%, at least about 96%, at least about 97%, at least about 98%, at least about 99%, or about 100% less CD14⁺ immunoresponsive cells compared to a sample.

16. The method of claim 15, wherein the composition comprises at least about 80% less CD14⁺ immunoresponsive cells compared to the sample.

17. The method of claim 1, wherein the composition comprises at least about 1%, at least about 5%, at least about 10%, at least about 15%, or at least about 20% of CD14⁺ immunoresponsive cells over the total number of cells.

18. The method of claim 17, wherein the composition comprises at least about 5% of CD14⁺ immunoresponsive cells over the total number of cells.

19. The method of claim 1, wherein the reagent activating and/or stimulating the immunoresponsive cells comprises an anti-CD3 antibody or an antigen-binding fragment thereof activating the immunoresponsive cell.

20. The method of claim 1, wherein the reagent activating and/or stimulating the immunoresponsive cells comprises an anti-CD28 antibody or an antigen-binding thereof.

21. The method of claim 1, wherein the reagent activating and/or stimulating the immunoresponsive cells comprises an anti-CD3 antibody or an antigen-binding fragment thereof activating the immunoresponsive cell and an anti-CD28 antibody or an antigen-binding thereof.

22. The method of any one of claims 19-21, wherein the reagent is bound to a magnetic particle.

23. The method of claim 1, wherein the immunoresponsive cells are T cells.

24. The method of claim 23, wherein the T cells are selected from the group consisting of cytotoxic T lymphocytes (CTL), γδ T cells, tumor-infiltrating lymphocytes (TIL), regulatory T cells, and Natural Killer T (NKT) cells.

25. The method of claim 23, wherein the T cells are CD8⁺ T cells.

26. The method of claim 23, wherein the T cells are CD4⁺ T cells.

27. The method of claim 25, wherein the CD8⁺ T cells are CD4 independent.

28. The method of claim 1, wherein the immunoresponsive cell are CD14⁻ cells.

29. The method of claim 1, wherein the immunoresponsive cells are obtained from a sample selected from the group consisting of a blood sample, a blood-derived sample, an apheresis product, a leukapheresis product, a whole blood sample, a peripheral blood mononuclear cells (PBMCs) sample, a leukocytes sample, a bone marrow sample, a thymus sample, a tissue biopsy sample, a tumor sample, a leukemia sample, a lymphoma sample, a lymph node sample, a gut associated lymphoid tissue sample, a mucosa associated lymphoid tissue sample, a spleen sample, a liver sample, a lung sample, a stomach sample, an intestine sample, a colon sample, a kidney sample, a pancreas sample, a breast sample, a bone sample, a prostate sample, a cervix sample, a testes sample, an ovaries sample, a tonsil sample, and a combination thereof.

30. The method of claim 29, wherein the sample is an apheresis product.

31. The method of claim 29, wherein the sample is a leukapheresis product.

32. The method of claim 1, wherein the immunoresponsive cells are obtained from a sample of a subject having acute myeloid leukemia (AML).

33. The method of claim 1 further comprising a density-based cell separation.

34. The method of 33, wherein the density-based cell separation allows purification of peripheral blood mononuclear cells (PBMCs).

35. The method of claim 1, wherein the antigen-recognizing receptor targets an antigen.

36. The method of claim 35, wherein the antigen is a tumor antigen or a pathogen antigen.

37. The method of claim 36, wherein the tumor antigen is selected from the group consisting of CD19, CD70, ILIRAP, ABCG2, AChR, ACKR6, ADAMTS13, ADGRE2 (EMR2), ADORA3, ADRA1D, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LlCAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC1I, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSFlB, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

38. The method of any one of claims 1-37, wherein the antigen-recognizing receptor is a chimeric receptor, a T cell receptor (TCR), or a TCR like fusion molecule.

39. The method of claim 38, wherein the antigen-recognizing receptor is a chimeric receptor.

40. The method of claim 39, wherein the chimeric receptor is a chimeric antigen receptor (CAR).

41. The method of claim 40, wherein the CAR comprises an extracellular antigen-binding domain that binds to the antigen, and an intracellular signaling domain that is capable of delivering an activation signal to the cell.

42. The method of claim 41, wherein the intracellular signaling domain of the CAR comprises a CD3ζ polypeptide.

43. The method of claim 42, wherein the CD3ζ polypeptide is a native CD3ζ polypeptide or a modified CD3ζ polypeptide.

44. The method of claim 43, wherein the modified CD3ζ polypeptide comprises a native ITAM1, an ITAM2 variant consisting of two loss-of-function mutations, and an ITAM3 variant consisting of two loss-of-function mutations.

45. The method of claim 41, wherein the intracellular signaling domain of the CAR further comprises at least one costimulatory signaling region.

46. The method of claim 45, wherein the at least one costimulatory signaling region comprises at least an intracellular domain of a co-stimulatory molecule or a portion thereof.

47. The method of claim 46, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

48. The method of claim 41, wherein the CAR comprises a transmembrane domain.

49. The method of claim 39, wherein the chimeric receptor is a chimeric co-stimulating receptor (CCR).

50. The method of claim 49, wherein the CCR comprises an extracellular antigen-binding domain that binds to the second antigen and an intracellular domain that is capable of delivering a costimulatory signal to the cell but does not alone deliver an activation signal to the cell.

51. The method of claim 50, wherein the intracellular domain of the CCR comprises at least an intracellular domain of a co-stimulatory molecule or a portion thereof.

52. The method of claim 51, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

53. The method of claim 38, wherein the antigen-recognizing receptor is a TCR like fusion molecule.

54. The method of claim 53, wherein the TCR like fusion molecule comprises i) a first antigen-binding chain comprising an antigen-binding fragment of a heavy chain variable region (V_{H}) of an antibody; and ii) a second antigen-binding chain comprising an antigen-binding fragment of a light chain variable region (V_{L}) of the antibody; wherein the first and second antigen-binding chains a) each comprise a TRAC polypeptide or a TRBC polypeptide, and b) bind to the first antigen, wherein the TCR like fusion molecule binds to the first antigen in an HLA-independent manner.

55. The method of claim 54, wherein at least one of the TRAC polypeptide and the TRBC polypeptide is endogenous.

56. The method of claim 54, wherein the antigen-binding chain comprises an antigen-binding fragment of a V_{H} of an antibody and a TRBC polypeptide, and the second antigen-binding chain comprises an antigen-binding fragment of a V_{L} of the antibody and a TRAC polypeptide.

57. The method of claim 54, wherein the antigen-binding chain comprises an antigen-binding fragment of a V_{H} of an antibody and a TRAC polypeptide, and the second antigen-binding chain comprises an antigen-binding fragment of a V_{L} of the antibody and a TRBC polypeptide.

58. The method of claim 54, wherein the first and second antigen binding chains are capable of associating with a CD3ζ polypeptide.

59. The method of claim 58, wherein the first and second antigen binding chains, upon binding to the first antigen, are capable of activating the CD3ζ polypeptide.

60. A composition comprising the immunoresponsive cell obtained by the method of any one of claims 1-59.

61. The composition of claim 60, which is a pharmaceutical composition further comprising a pharmaceutically acceptable excipient.

62. A method of reducing tumor burden in a subject, the method comprising administering to the subject an effective amount of the immunoresponsive cell produced by the method of any one of claims 1-59 or the composition of claim 60 or 61.

63. The method of claim 62, wherein the method reduces the number of tumor cells, reduces tumor size, and/or eradicates the tumor in the subject.

64. A method of preventing and/or treating a neoplasm or a tumor in the subject, administering to the subject an effective amount of the immunoresponsive cell produced by the method of any one of claims 1-59 or the composition of claim 60 or 61.

65. The method of any one of claims 62-64, wherein the neoplasm or tumor is cancer.

66. The method of any one of claims 62-65, wherein the neoplasm or tumor is a solid tumor.

67. The method of any one of claims 62-65, wherein the neoplasm or tumor is a blood cancer.

68. The method of any one of claims 62-65, and 67, wherein the neoplasm or tumor is a myeloid disorder.

69. The method of claim 68, wherein the myeloid disorder is selected from the group consisting of myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

70. The method of claim 69, wherein the myeloid disorder is acute myeloid leukemia (AML).

71. A method of preventing and/or treating a pathogen infection in a subject, the method comprising administering to the subject an effective amount of the immunoresponsive cell produced by the method of any one of claims 1-59 or the composition of claim 60 or 61.

72. A method of preventing and/or treating an autoimmune disease in a subject, the method comprising administering to the subject an effective amount of the immunoresponsive cell produced by the method of any one of claims 1-59 or the composition of claim 60 or 61.

73. A method of preventing and/or treating an infectious disease in a subject, the method comprising administering to the subject an effective amount of the immunoresponsive cell produced by the method of any one of claims 1-59 or the composition of claim 60 or 61.

74. A kit comprising the immunoresponsive cell produced by the method of any one of claims 1-59 or the composition of claim 60 or 61.

75. The kit of claim 74, wherein the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.

76. The composition of claim 60 or 61 or the kit of claim 74 or 75 for use in reducing tumor burden, treating and/or preventing a neoplasm or a tumor, preventing and/or treating a pathogen infection, preventing and/or treating an autoimmune disease, and/or preventing and/or treating an infectious disease, in a subject.


SEQUENCE LISTING (58 sequences)

SEQ ID NO: 1 - protein, 15 aa, synthetic construct
  REGION 1..15: note = Description of sequence: Linker
GGGGSGGGGSGGGGS

SEQ ID NO: 2 - protein, 19 aa, synthetic construct
  REGION 1..19: note = Description of sequence: Linker
GGGGSGGGGSGGGSGGGGS

SEQ ID NO: 3 - protein, 24 aa, synthetic construct
  REGION 1..24: note = Description of sequence: Linker
GGGGSGGGGSGGGGSGGGSGGGGS

SEQ ID NO: 4 - protein, 29 aa, synthetic construct
  REGION 1..29: note = Description of sequence: Linker
GGGGSGGGGSGGGGSGGGGSGGGSGGGGS

SEQ ID NO: 5 - protein, 5 aa, synthetic construct
  REGION 1..5: note = Description of sequence: Linker
GGGGS

SEQ ID NO: 6 - protein, 10 aa, synthetic construct
  REGION 1..10: note = Description of sequence: Linker
GGGGSGGGGS

SEQ ID NO: 7 - protein, 220 aa, Homo sapiens
  REGION 1..220: note = Description of sequence: transmembrane domain
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 164 aa, Homo sapiens
  REGION 1..164: note = Description of sequence: intracellular signaling domain
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 112 aa, Homo sapiens
  REGION 1..112: note = Description of sequence: CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 22 aa, Homo sapiens
  REGION 1..22: note = Description of sequence: intracellular signaling domain
QNQLYNELNLGRREEYDVLDKR

SEQ ID NO: 11 - DNA, 66 bp, synthetic construct
  misc_feature 1..66: note = Description of sequence: exemplary coding sequence of intracellular signaling domain
  source 1..66: mol_type = other DNA
cagaaccagctctataacgagctcaatctaggacgaagagaggagtacgatgttttggacaagaga

SEQ ID NO: 12 - protein, 22 aa, synthetic construct
  REGION 1..22: note = Description of sequence: ITAM1 variant
QNQLFNELNLGRREEFDVLDKR

SEQ ID NO: 13 - DNA, 66 bp, synthetic construct
  misc_feature 1..66: note = Description of sequence: exemplary coding sequence of ITAM1 variant
  source 1..66: mol_type = other DNA
cagaaccagctctttaacgagctcaatctaggacgaagagaggagttcgatgttttggacaagaga

SEQ ID NO: 14 - protein, 23 aa, Homo sapiens
  REGION 1..23: note = Description of sequence: Native ITAM1
QEGLYNELQKDKMAEAYSEIGMK

SEQ ID NO: 15 - DNA, 69 bp, synthetic construct
  misc_feature 1..69: note = Description of sequence: exemplary coding sequence of native ITAM1
  source 1..69: mol_type = other DNA
caggaaggcctgtacaatgaactgcagaaagataagatggcggaggcctacagtgagattgggatgaaa

SEQ ID NO: 16 - protein, 23 aa, Homo sapiens
  REGION 1..23: note = Description of sequence: ITAM2 variant
QEGLFNELQKDKMAEAFSEIGMK

SEQ ID NO: 17 - DNA, 69 bp, synthetic construct
  misc_feature 1..69: note = Description of sequence: exemplary coding sequence of ITAM2 variant
  source 1..69: mol_type = other DNA
caggaaggcctgttcaatgaactgcagaaagataagatggcggaggccttcagtgagattgggatgaaa

SEQ ID NO: 18 - protein, 22 aa, Homo sapiens
  REGION 1..22: note = Description of sequence: Native ITAM3
HDGLYQGLSTATKDTYDALHMQ

SEQ ID NO: 19 - DNA, 66 bp, synthetic construct
  misc_feature 1..66: note = Description of sequence: exemplary coding sequence of native ITAM3
  source 1..66: mol_type = other DNA
cacgatggcctttaccagggtctcagtacagccaccaaggacacctacgacgcccttcacatgcag

SEQ ID NO: 20 - protein, 22 aa, Homo sapiens
  REGION 1..22: note = Description of sequence: ITAM3 variant
HDGLFQGLSTATKDTFDALHMQ

SEQ ID NO: 21 - DNA, 66 bp, synthetic construct
  misc_feature 1..66: note = Description of sequence: exemplary coding sequence of ITAM3 variant
  source 1..66: mol_type = other DNA
cacgatggccttttccaggggctcagtacagccaccaaggacaccttcgacgcccttcacatgcag

SEQ ID NO: 22 - protein, 112 aa, synthetic construct
  REGION 1..112: note = Description of sequence: modified CD3 zeta polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 23 - DNA, 336 bp, synthetic construct
  misc_feature 1..336: note = Description of sequence: exemplary coding sequence of modified CD3 zeta polypeptide
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 24 - DNA, 123 bp, Homo sapiens
  misc_feature 1..123: note = Description of sequence: transmembrane domain exemplary coding sequence
  source 1..123: mol_type = genomic DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 25 - protein, 218 aa, Homo sapiens
  REGION 1..218: note = Description of sequence: intracellular signaling domain
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 26 - protein, 255 aa, Homo sapiens
  REGION 1..255: note = Description of sequence: co-stimulatory signaling region
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 27 - protein, 20 aa, Homo sapiens
  REGION 1..20: note = Description of sequence: human IL-2 signal sequence
MYRMQLLSCIALSLALVTNS

SEQ ID NO: 28 - protein, 20 aa, Mus musculus
  REGION 1..20: note = Description of sequence: mouse IL-2 signal sequence
MYSMQLASCVTLTLVLLVNS

SEQ ID NO: 29 - protein, 20 aa, Homo sapiens
  REGION 1..20: note = Description of sequence: human kappa leader sequence
METPAQLLFLLLLWLPDTTG

SEQ ID NO: 30 - protein, 20 aa, Homo sapiens
  REGION 1..20: note = Description of sequence: human CD8 leader sequence
METDTLLLWVLLLWVPGSTG

SEQ ID NO: 31 - protein, 21 aa, Homo sapiens
  REGION 1..21: note = Description of sequence: human CD8 signal peptide
MALPVTALLLPLALLLHAARP

SEQ ID NO: 32 - protein, 18 aa, Homo sapiens
  REGION 1..18: note = Description of sequence: truncated human CD8 signal peptide
MALPVTALLLPLALLLHA

SEQ ID NO: 33 - protein, 16 aa, Homo sapiens
  REGION 1..16: note = Description of sequence: human albumin signal sequence
MKWVTFISLLFSSAYS

SEQ ID NO: 34 - protein, 30 aa, Homo sapiens
  REGION 1..30: note = Description of sequence: human prolactin signal sequence
MDSKGSSQKGSRLLLLLVVSNLLLCQGVVS

SEQ ID NO: 35 - DNA, 4627 bp, Homo sapiens
  misc_feature 1..4627: note = Description of sequence: TRAC polypeptide gene
  source 1..4627: mol_type = genomic DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 36 - protein, 178 aa, Homo sapiens
  REGION 1..178: note = Description of sequence: TRAC polypeptide
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 37 - DNA, 534 bp, synthetic construct
  misc_feature 1..534: note = Description of sequence: TRAC polypeptide exemplary coding sequence
  source 1..534: mol_type = other DNA
gatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 38 - protein, 175 aa, Homo sapiens
  REGION 1..175: note = Description of sequence: TRBC1 polypeptide
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 39 - protein, 176 aa, Homo sapiens
  REGION 1..176: note = Description of sequence: TRBC1 polypeptide
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 40 - DNA, 528 bp, synthetic construct
  misc_feature 1..528: note = Description of sequence: TRBC1 polypeptide exemplary coding sequence
  source 1..528: mol_type = other DNA
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 41 - DNA, 1448 bp, Homo sapiens
  misc_feature 1..1448: note = Description of sequence: TRBC gene
  source 1..1448: mol_type = genomic DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 42 - DNA, 1489 bp, Homo sapiens
  misc_feature 1..1489: note = Description of sequence: TRBC gene
  source 1..1489: mol_type = genomic DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 43 - protein, 10 aa, synthetic construct
  REGION 1..10: note = Description of sequence: hinge/spacer region
IPNIQNPDPA

SEQ ID NO: 44 - DNA, 30 bp, synthetic construct
  misc_feature 1..30: note = Description of sequence: hinge/spacer region exemplary coding sequence
  source 1..30: mol_type = other DNA
attcccaatatccagaaccctgaccctgcc

SEQ ID NO: 45 - protein, 182 aa, Homo sapiens
  REGION 1..182: note = Description of sequence: CD3 gamma chain
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 46 - protein, 171 aa, synthetic construct
  REGION 1..171: note = Description of sequence: CD3 gamma chain variant
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 47 - protein, 127 aa, synthetic construct
  REGION 1..127: note = Description of sequence: CD3 gamma chain variant
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 48 - protein, 207 aa, synthetic construct
  REGION 1..207: note = Description of sequence: CD3 epsilon chain
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 49 - protein, 254 aa, Homo sapiens
  REGION 1..254: note = Description of sequence: human 4-1BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 50 - DNA, 762 bp, synthetic construct
  misc_feature 1..762: note = Description of sequence: 4-BBL exemplary coding sequence
  source 1..762: mol_type = other DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 51 - protein, 288 aa, Homo sapiens
  REGION 1..288: note = Description of sequence: CD 80 ligand
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 52 - DNA, 864 bp, synthetic construct
  misc_feature 1..864: note = Description of sequence: CD 80 ligand exemplary coding sequence
  source 1..864: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 53 - protein, 309 aa, synthetic construct
  REGION 1..309: note = Description of sequence: fusion polypeptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 54 - protein, 349 aa, synthetic construct
  REGION 1..349: note = Description of sequence: fusion polypeptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFRSKRSRLLHSDYMNMTPRRPGPTRKHYQPYAPPRDFAAYRKRGRKKLLYIFKQPFMRPVQTTQEEDGCSCRFPEEEEGGCEL

SEQ ID NO: 55 - protein, 119 aa, synthetic construct [sequence omitted]

SEQ ID NO: 56 - protein, 106 aa, synthetic construct [sequence omitted]

SEQ ID NO: 57 - protein, 11 aa, synthetic construct
  REGION 1..11: note = Description of sequence: hinge/spacer region
LEDLKNVFPPE

SEQ ID NO: 58 - DNA, 33 bp, synthetic construct
  misc_feature 1..33: note = Description of sequence: hinge/spacer region exemplary coding sequence
  source 1..33: mol_type = other DNA
ctggaggatctgaaaaacgtgttccctcctgaa